(19) United States
(12) Patent Application Publication
(10) Pub. No.: US 2026/0216124 A1
(43) Pub. Date: Jul. 30, 2026

(54) COMPOUNDS, PROCESS OF PRODUCTION OF COMPOUNDS, ENRICHED EXTRACT, ENRICHED EXTRACT ACTIVE FRACTIONS, ENRICHED EXTRACT PROCESS OF PRODUCTION, METHOD FOR SELECTING VEGETAL BIOMASS FOR ENRICHED EXTRACT PRODUCTION, COMPOSITION AND USE FOR TREATMENT OF IMMUNOLOGICAL DISORDERS

(71) Applicant: ACHÉ LABORATÓRIOS FARMACÊUTICOS S.A., Guarulhos (BR)

(72) Inventors: Cristiano Ruch Werneck Guimaraes, Sao Paulo (BR); Carlos Eduardo Vitor, Sao Paulo (BR); Lisandra Ravanelli Pessa, Sao Paulo (BR); Romulo Dragani Reis, Sao Paulo (BR); Alessandra Mascarello, Sao Paulo (BR); Fernando Henrique De Souza Gama, Sao Paulo (BR)

(21) Appl. No.: 19/578,332

(22) Filed: Mar. 25, 2026

Related U.S. Application Data
(63) Continuation-in-part of application No. 16/486,683, filed on Feb. 19, 2018.

(30) Foreign Application Priority Data
Feb. 17, 2017 (BR) BR1020170033180

(51) Int. Cl. A61K 31/352; A61K 31/191; A61K 31/215; C07C 59/105; C07C 69/738; C07D 311/94

(52) U.S. Cl. CPC A61K 31/352; A61K 31/191; A61K 31/215; C07C 59/105; C07C 69/738; C07D 311/94; A61K 2236/15; A61K 2236/17; A61K 2236/31; A61K 2236/53; C07C 2601/10

(57) ABSTRACT

Isolated compounds and extracts with immunosuppressive activity and methods of using and producing these compounds and extracts are provided by a process using plants of the genus Stachytarpheta. The compounds and extracts of the disclosure may also be used in a method of treating an immunological disorder by administering to a patient in need an effective amount of the compositions or extracts and/or standardized fractions containing the compounds and extracts. The present disclosure thus relates to the fields of pharmacy, medicine, and chemistry.

### CROSS-REFERENCE TO RELATED APPLICATIONS

This application is a continuation-in-part application of U.S. application Ser. No. 16/486,683, having a filing date of Aug. 16, 2019, which was a National Stage application of International Application No. PCT/BR2018/050037, filed Feb. 19, 2018, and also claims the benefit of Brazilian Application No. BR1020170033180, filed Feb. 17, 2017, all of said applications being incorporated in their entirety herein by reference.

### FIELD OF THE INVENTION

The following invention describes novel and inventive isolated compounds and extracts with immunosuppressive activity and a process of producing compounds from ipolamiide and extracts, from plants of the genus Stachytarpheta. In this way, the present application also describes novel and inventive compositions and their use for the treatment of immunological disorders. The present invention is in the fields of pharmacy, medicine and chemistry.

### BACKGROUND OF THE INVENTION

### Immunological Disorders

Immunological disorders can be considered as any imbalance or malfunction of the immune system. This system is primarily responsible for assisting in the defense of the body against external or unknown agents through antibodies that recognize and fight harmful antigens.

Autoimmune diseases are an of immunological disorders in which the organism starts to produce antibodies against their own molecules, making no distinction between endogenous and exogenous agents. In such cases, medicaments with immunosuppressive activity would be highly demanded to help patients relieve the symptoms caused by this type of disorder.

Autoimmune diseases, except for rheumatoid arthritis and autoimmune thyroiditis, are individually rare, but together they affect approximately 5% of the population of the western countries. Their etiology is not fully understood. In organ-specific and systemic autoimmune diseases, it is observed a loss of the capacity of the immune system to distinguish what is self from what is not self. This ability, called self-tolerance, is maintained in the immunocompetent B and T cells by both central and peripheral mechanisms. The loss of self-tolerance may have intrinsic or extrinsic causes. Environmental factors such as bacterial and viral infections, exposure to physical and chemical agents such as UV, pesticides and drugs are examples of extrinsic causes. Intrinsic causes, that is, related to characteristics of the individual itself, are usually associated with polymorphisms of histocompatibility molecules, components of innate immunity, such as the complement system and Toll-like receptors, components of acquired immunity as regulatory lymphocytes and cytokines, in addition to hormonal factors which are under genetic control.

The therapeutic strategy in autoimmune diseases mainly consists in suppressing the immunological system using immunosuppressants, which act on the inhibition of the early stages of development of immunity. This therapy does not perform a selective immunosuppression, which led to the development of a variety of antibodies. Biological agents may be employed to inhibit the effect of cytokines, as occurs with anti-cytokine monoclonal antibodies or the use of soluble receptors that bind to the cytokine and block their effects on target cells. The cytokines are also used in biological therapy through analogous recombinant proteins that mimic the effect of the original cytokine. The main therapeutic targets in anti-cytokines therapy are the pro-inflammatory cytokines interleukin-1 (IL-1), TNFα and IL-6, and the main cytokine agonist therapy is performed with the use of type I interferons (IFN). In the type I interferon family, recombinant proteins of IFNα and of IFNβ are used in clinical practice, primarily for the treatment of viral hepatitis (hepatitis virus B and C) and of multiple sclerosis, respectively. For the latter, the proposed mechanism of action is the antagonism that the INFβ exerts against IFNγ, which has great importance in the physiopathology of multiple sclerosis. The IFNα has also been used in the treatment of muco-cutaneous, ocular and neurological manifestations of Behçet's disease and Churg-Strauss syndrome. Fontolizumab is a humanized anti-IFNγ agent, which has been evaluated in patients with abnormalities in dendritic cells with good results.

Monoclonal antibodies have advantages in the treatment of autoimmune diseases when compared with conventional therapies as they are a targeted therapy, with specificity and high selectivity. However, they are used as a second line of treatment when there is no effectiveness in the control of autoimmune diseases with conventional therapies. In addition to restrictions on the efficacy, monoclonal antibodies still have several limitations related to cost and accessibility. Several autoimmune diseases still lack effective treatments, with good tolerability by patients.

Natural products have been used for centuries in the treatment of different diseases. Recently, major efforts have been made in the development of new research of herbal products with immunosuppressive effects. For example, several clinical trials performed in the United States have already shown significant benefits of T. wilfordii extract in patients with rheumatoid arthritis. Although there are several species and their active constituents with mechanisms of action described, there is still a vast field to be explored regarding in vitro and in vivo investigations and future clinical trials in immunologically based diseases. These natural products should be formulated in appropriate pharmaceutical compositions in order to ensure the effectiveness of the treatment of autoimmune diseases.

An autoimmune disease for which, so far, there is no effective medication, much less a medication option obtained from natural extracts, can be exemplified by vitiligo. Vitiligo is commonly associated with loss of functional melanocytes and is considered the most common acquired depigmentation disorder in humans, affecting at least 0.5% of the world population. It is characterized by the development of white maculae, resulting from the loss of epidermal melanocytes, which can result in cellular destruction through a specific cytotoxic immune response to melanocytes and in damage to the adhesion system thereof.

Multiple mechanisms have been associated with vitiligo such as genetic predisposition, environmental activations, metabolic anomalies and changes in the immune and inflammatory responses. In addition, conditions such as exposure to ultraviolet radiation and oxidative stress are known to aggravate this condition.

Due to the lack of specific knowledge about the initial onset of the disease, several studies try to elucidate the biological pathways involved in this pathogenesis. Most of them indicate the complexity and the challenges related to such disease, being very difficult to find an efficient treatment. We must not forget that most immunological disorders have an important social impact, causing a high level of psychological stress for the patients. To date, there is no intervention capable of delivering the cure of vitiligo.

In this way, it becomes eminent the need to identify new compounds capable of promoting immunosuppressive activity with long-term effect. Thus, the present invention addresses this gap in the treatment of immunological disorders through novel compounds which, when isolated, demonstrate immunosuppressive activity and active extracts comprising groups of active compounds, which are obtained in an unique and inventive manner.

Highlighting the complexity and lack of scientific knowledge about vitiligo, it was believed so far that the structure of ipolamiide in its intact form could be associated with immunosuppressive activity, relevant to the treatment of patients with vitiligo. However, in the present invention we demonstrate that this activity results from specific derivatives of ipolamiide and of the extracts comprising such compounds, preferably obtained by the unique and inventive processes of production described herein. Both compounds and extracts present immunosuppressive activity confirmed experimentally. This activity is demonstrated herein by blocking the activation of CD8+ T cells and reduction of IFN-γ secretion. This mechanism has been shown to be promising for vitiligo in view of recent clinical findings related to the quantification of these components in the skin of patients. Specifically, high concentrations of CD8+ T cells and IFN-γ are linked to the apoptosis of melanocytes and, therefore, the modulation of these is a promising mechanism of action.

From what can be deduced from the researched literature, no documents were found anticipating or suggesting the teachings of the present invention, so that the technical solution proposed herein has novelty and inventive activity in view of the state of the art.

### SUMMARY OF THE INVENTION

The present invention describes novel and inventive compounds derived from ipolamiide, which have immunosuppressive activity. Therefore, they can be used for the treatment of immunological disorders. Additionally, the present invention describes vegetal extracts enriched with said compounds, derived from ipolamiide, obtained through an unique process of production, also having immunosuppressive activity.

It is, therefore, an object of the present invention ipolamiide derivatives comprising compounds of the general formula:

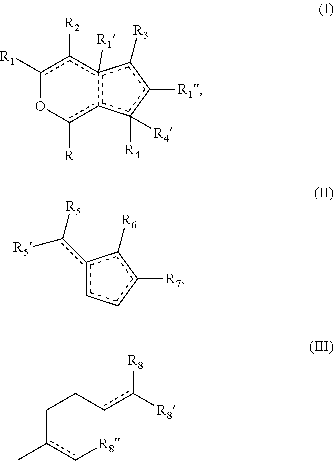

wherein R corresponds to H, OH, OGlyc (Glycoside); R₁, R_{1′}, R_{1″} correspond to H, OH; R₂ corresponds to H, COOH, COOCH₃, CH₃, CHO; R₃ corresponds to H, OH, CH₃; R₄, R_{4′} correspond to H, OH, CH₂OH, CH₃; R₅, R_{5′} correspond to H, CH₃, COOCH₃, CHO, CH₂OH; Re corresponds to CHO, COOH, COOCH₃; R₇ corresponds to H, CH₃; R₈, R_{8′}, R_{8″} correspond to CHO, CH₃, CH₂OH, COOH and the dashed bonds represent single (C—C) or double (C═C) bonds between carbons (up to two double bonds per structure). In addition, R₁ may be absent or correspond to H or OH.

In a preferred embodiment, at least one preferred compound of the present invention may be selected from the group comprising the following structures:

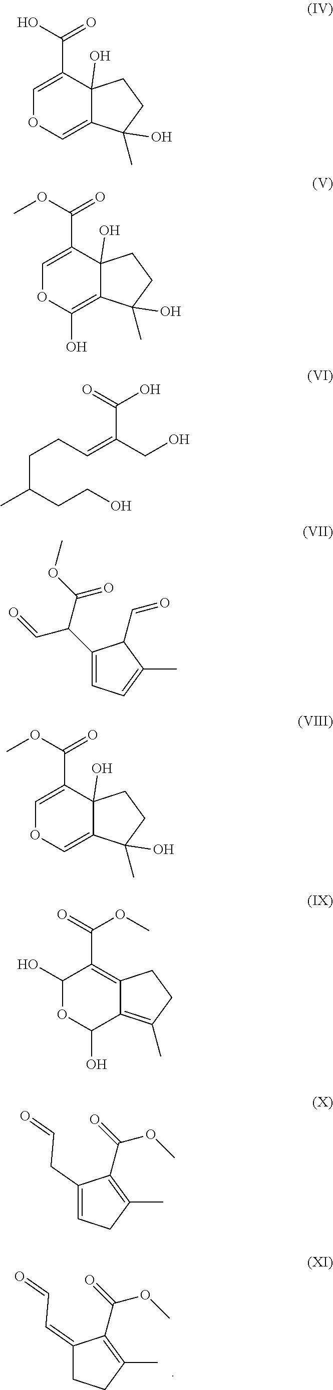

In this preferred embodiment, the compound of general formula (I) comprises the compounds of formula (IV), (V), (VIII) and (IX), the compound of general formula (II) comprises the compound of formula (VII), (X) and (XI) and the compound of general formula (III) comprises the compound of formula (VI).

It is also an object of the present invention a method of treatment of immunological disorders, comprising administering to a patient a compound of general formula (I), (II) and/or (III), in sufficient amount to provide immunosuppressive effect. In a preferred embodiment, the method of treatment is intended for the treatment of vitiligo.

We can, thus, also consider the use of at least one compound of general formula (I), (II) and/or (III) in a composition for treatment of immunological disorders.

Furthermore, it is an object of the present invention a pharmaceutical composition for the treatment of immunological disorders, comprising at least one compound selected among the groups comprising the compounds of general formula:

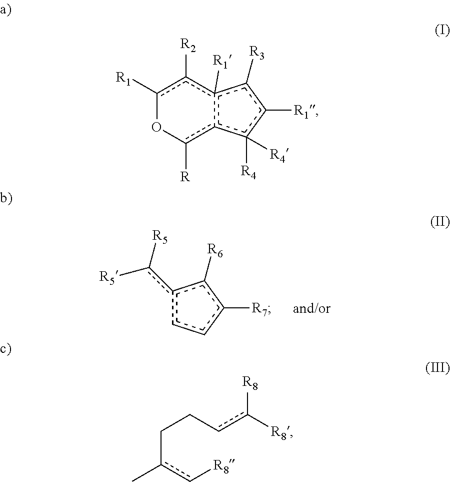

wherein R corresponds to H, OH, OGlyc (Glycoside); R₁, R_{1′}, R_{1″} correspond to H, OH; R₂ corresponds to H, COOH, COOCH₃, CH₃, CHO; R₃ corresponds to H, OH, CH₃; R₄, R_{4′} correspond to H, OH, CH₂OH, CH₃; R₅, R_{5′} correspond to H, CH₃, COOCH₃, CHO, CH₂OH; Re corresponds to CHO, COOH, COOCH₃; R₇ corresponds to H, CH₃; R₈, R_{′}, R_{8″} correspond to CHO, CH₃, CH₂OH, COOH and the dashed bonds represent single (C—C) or double (C═C) bonds between carbons (up to two double bonds per structure); and
d) pharmaceutically acceptable vehicle.

In an optional embodiment, the composition of the present invention further comprises the ipolamiide compound.

In an optional embodiment, the composition of the present invention may further comprise at least one of the following compounds:

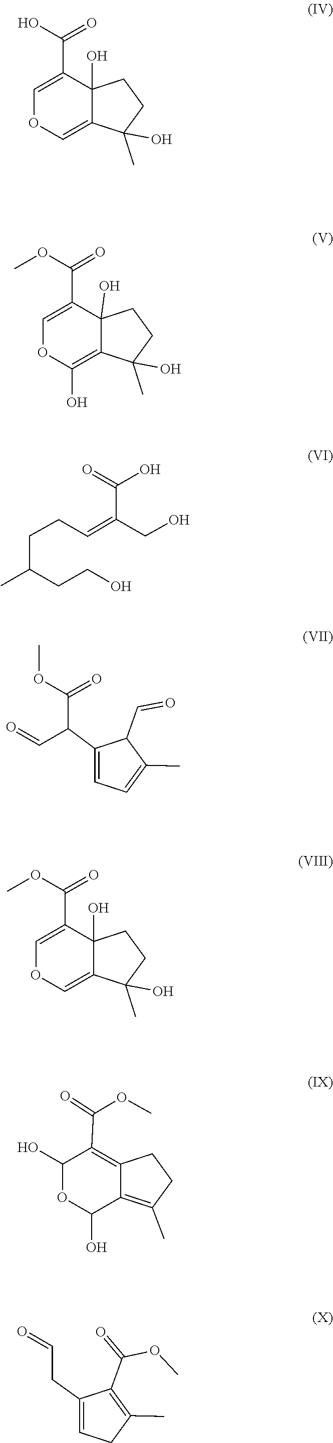

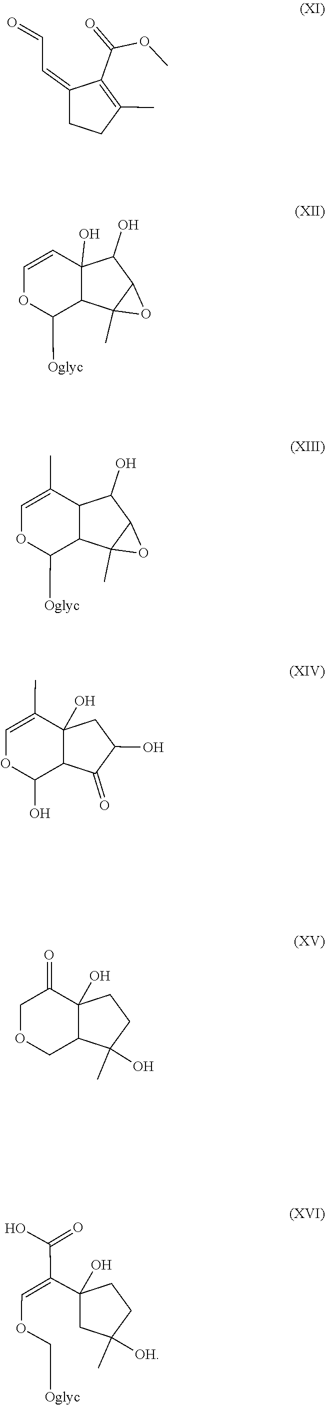

Additionally, the present invention describes the process for production of the compounds of general formula (I), (II) and/or (III), comprising the step of subjecting at least one ipolamiide compound to at least one heating step at high temperatures, in the presence of at least one suitable solvent for a sufficient time to obtain the compounds of the general formulas (I), (II) and/or (III).

In a preferred embodiment, the high temperatures of the present invention comprise temperatures above 35° C., more preferably between 35° C. and 165° C.

In a preferred embodiment, the process of production of the compounds of general formula (I), (II), and/or (III) comprises subjecting at least one ipolamiide compound to at least one hydrolysis and/or solvolysis step. Even more ideally, at least one ipolamiide compound is subjected to an acid hydrolysis step. Optionally, at least one ipolamiide compound is subjected to an alkaline/basic hydrolysis step.

As previously mentioned, we verified that isolated ipolamiide does not demonstrate immunosuppressive activity. On the other hand, certain groups of compounds derived from ipolamiide have such activity. At the same time, we have also specified the advantages of obtaining herbal medicines for the treatment of diseases, since these compound production systems allow a series of productive interactions between the components of the plant and the active compounds, often even synergistically. Thus, to additionally obtain an herbal medicine comprising such active compounds, we have developed an unique production process which allows to obtain an extract enriched with compounds of interest. As described below, the extract production process of the present invention comprises unique steps that lead to extracts enriched with the ipolamiide derivatives with immunosuppressive activity. We verified the relevance of preselecting input vegetal biomasses containing between 2.5% and 3.5% of ipolamiide, resulting in an extract enriched with ipolamiide and compounds derived from ipolamiide from about 1% to about 20%, preferably from about 8.5% to about 11.5% of content of ipolamiide and derivatives.

As previously presented, the vegetal biomass containing this compound will be used as starting material for the production process of the extract. Only with the production process of the present invention it is possible to obtain an extract enriched with specific compounds derived from ipolamiide. This enriched extract, further, presents immunosuppressive activity.

It is, therefore, an additional object of the present invention a process for production of extract enriched with compounds derived from ipolamiide, comprising essentially the steps of:
a) selecting input vegetal biomass with a content of ipolamiide between 2.5% and 3.5% obtained from plants of the genus Stachytarpheta;
b) submitting the selected biomass from a) to oven drying at temperature between 40 to 80° C., until obtaining the humidity stabilization between 10 to 12%;
c) milling the vegetal biomass;
d) performing the extraction of the vegetal biomass through the steps of:
i. heating of the vegetal biomass at a temperature between 70 to 100° C., with constant stirring;
ii. maceration of the vegetal biomass at room temperature;
iii. heating of the vegetal biomass with temperature between 70 to 100° C.

In a preferred embodiment, the process for production of the present invention further comprises the steps of:
iv. filtering and concentration of the extract;
v. drying in Spray Dryer, during 1 to 60 seconds, with inlet temperature between 155 and 165° C. and outlet temperature between 85 to 95° C., coupled to a dehumidifier.

In a preferred embodiment, the process for extraction of the present invention is an aqueous or hydroalcoholic process, even more preferably aqueous process.

Therefore, the process for production of the present invention allows to obtain a standardized extract enriched with compounds derived from ipolamiide, preferably with a yield of about 8% to about 10%.

It is, therefore, an additional object of the present invention the extract enriched with compounds derived from ipolamiide obtained by the above-mentioned procedure. The standardized extract enriched with compounds derived from ipolamiide of the present invention comprises, preferably, the compounds of formula (I), (II) and/or (III).

The vegetal biomass of the present invention comprises all parts of plants of the genus Stachytarpheta. Preferably, the vegetal biomass comprises the aerial parts of the plants, more preferably, the leaves.

In a preferred embodiment, the input vegetal biomass comprises at least one vegetal biomass with uniform content of ipolamiide between 2.5% and 3.5%. In an optional embodiment, the input vegetal biomass comprises more than one vegetal biomass, wherein the different vegetal biomasses have different contents of ipolamiide independently, but together achieve an uniform content of ipolamiide (between 2.5% and 3.5%).

In another preferred embodiment, the actual content of ipolamiide in the input vegetal biomass can be used as a parameter for predicting the theoretical content of ipolamiide and derivatives in the extract obtained. This prediction can be accomplished by a method comprising the step of applying Equation I to some parameters obtained experimentally to find the ideal proportions of ipolamiide in the input vegetal biomass, which preferably projects the content of ipolamiide and derivatives in the extract from 8.5% to 11.5% of. The Equation I is defined below:

$$\% Theoretical content of ipolamiide and derivatives in the extract = \% Actual content of ipolamiide in the vegetal biomass \times DER / ( < actual content of ipolamiide and derivatives in the extract / actual content of ipolamiide in the input vegetal biomass > ) \pm standard deviation . \qquad ( Equation I )$$

In this way, it is possible to predict the theoretical content of ipolamiide and derivatives in the extract from the actual content of ipolamiide in the input vegetal biomass. Preferably, the ratio between the actual content of ipolamiide and derivatives in the extract/content of ipolamiide in the input vegetal biomass is between about 3.0 and about 3.5.

In an embodiment even more preferred, the plants of the present invention comprise Stachytarpheta cayennensis.

It is, therefore, an additional object of the present invention the use of standardized extract enriched with compounds derived from ipolamiide, obtained from plants of the genus Stachytarpheta for the manufacture of a medicament with immunosuppressive activity.

It is, therefore, an additional object of the present invention at least one active fraction of extract enriched with compounds derived from ipolamiide. Preferably, at least one fraction comprises at least one compound derived from ipolamiide of formula (I), (II) and/or (III).

In an optional embodiment, the active fraction of enriched extract further comprises ipolamiide.

It is, therefore, an additional object of the present invention the use of at least one standardized fraction enriched with compounds derived from ipolamiide, obtained from plants of the genus Stachytarpheta for the manufacture of a medicament with immunosuppressive activity.

These and other objects of the invention will be readily appreciated by those skilled in the art and by the companies having interests in the segment, and will be described in sufficient detail for its reproduction in the following description.

### DETAILED DESCRIPTION OF THE FIGURES

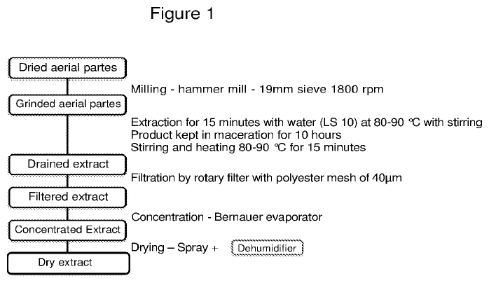

FIG. 1—Summary flowchart describing the production process of the active extract enriched with ipolamiide derivatives obtained from Stachytarpheta cayennensis.

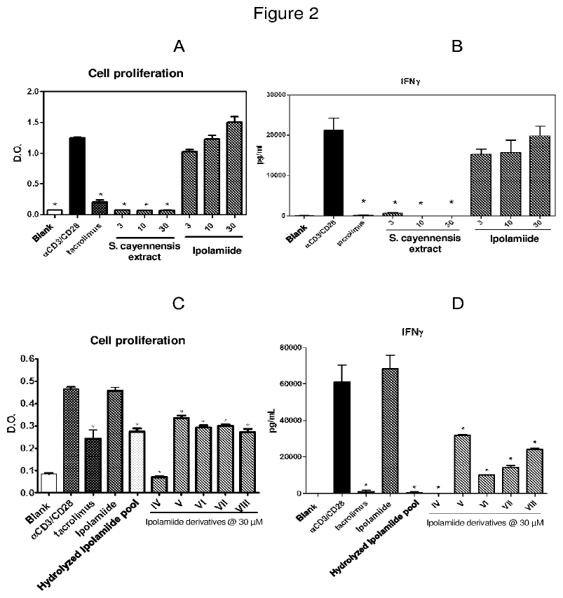

FIG. 2—Effect of the aqueous extract of Stachytarpheta cayennensis (3, 10 and 30 μM, concentration expressed in ipolamiide) and isolated ipolamiide (3, 10 and 30 μM) on the proliferation of CD8+ T cells activated by αCD3/CD28 (A) and IFNγ production (B). The effect of the pool of compounds (IV to VIII) and the five novel isolated compounds (IV to VIII) generated after the acid hydrolysis of ipolamiide (30 μM) was also evaluated in the same experiments, proliferation of CD8+ T cells activated by αCD3/CD28 (C) and IFNγ production (D). Tacrolimus (0.5 UM) was used as the positive control for all experiments. The data are the mean±SD of three replicates.

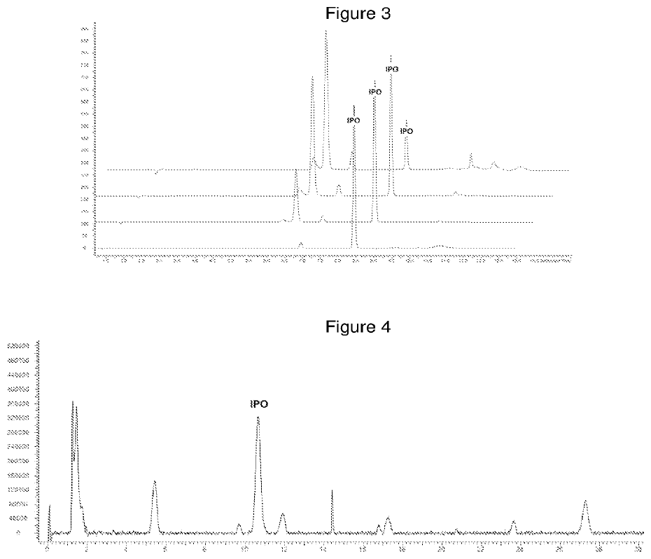

FIG. 3—Effect of the acid hydrolysis of ipolamiide on the formation of its derivatives. Chromatogram of intact ipolamiide (blue); Ipolamiide hydrolyzed at 0.1N HCl at 40° C. for 1 h (green); Ipolamiide hydrolyzed at 0.1N HCl at 40° C. for 2 h (red); Ipolamiide hydrolyzed at 0.1N HCl at 40° C. for 5 h (magenta). IPO=Ipolamide.

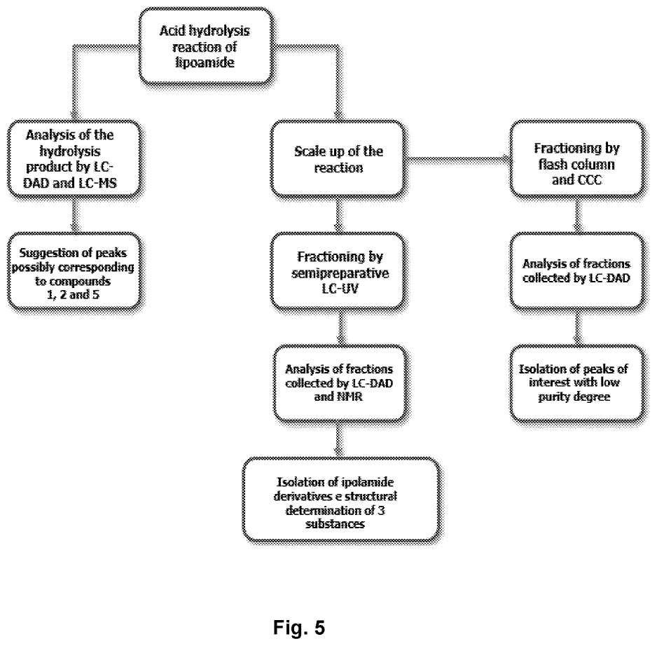

FIG. 4—Chromatogram of the Stachytarpheta cayennensis extract obtained from the production process claimed herein. The figure illustrates the ipolamiide marker and its specific derivatives at retention times: 5.5; 9.7; 12.0; 14.3; 17.3 min. IPO=Ipolamiide.

FIG. 5 shows the experimental design of the AGE 01-18 project. in accordance with the present invention.

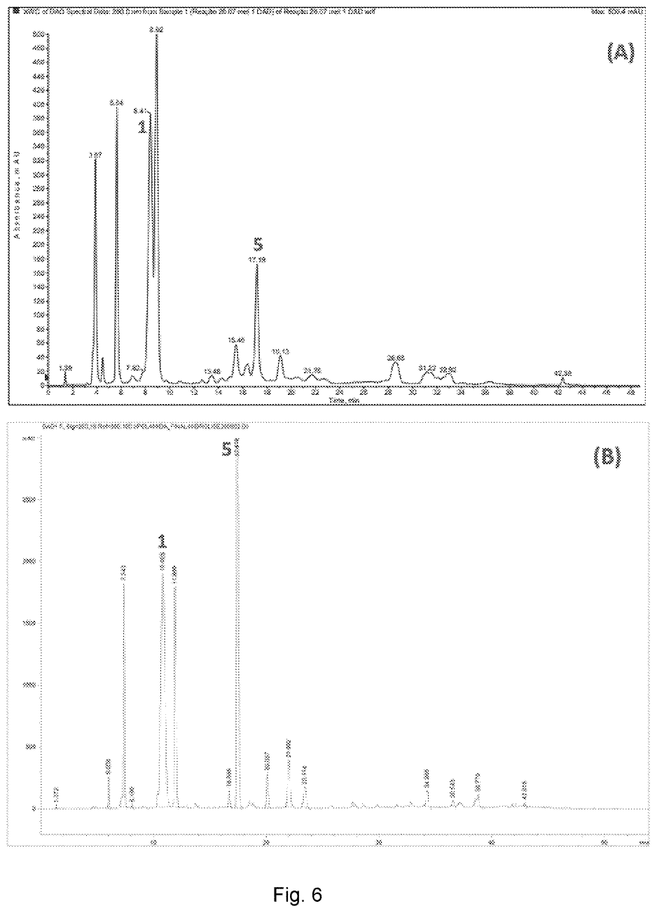

FIG. 6 shows a comparison between the chromatographic profiles of the reaction product of the ipolamide acid hydrolysis (A): Chromatogram (LC-DAD, 260 nm) of the ipolamide hydrolysis product obtained in this study. (B): Chromatogram (LC-DAD, 260 nm) of the ipolamide hydrolysis product, presented in the studies conducted by Applicant.

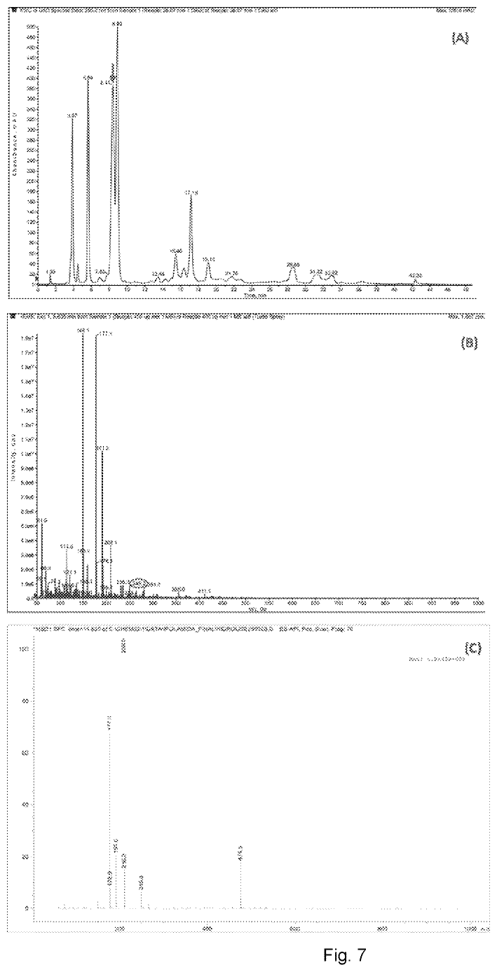

FIG. 7 shows (A): Chromatogram of the ipolamide hydrolysis product (LC-DAD, 260 nm). (B): MS spectrum of the peak with Tr=8.41 min. (C): MS spectrum of the compound 1, presented in the studies conducted by Applicant.

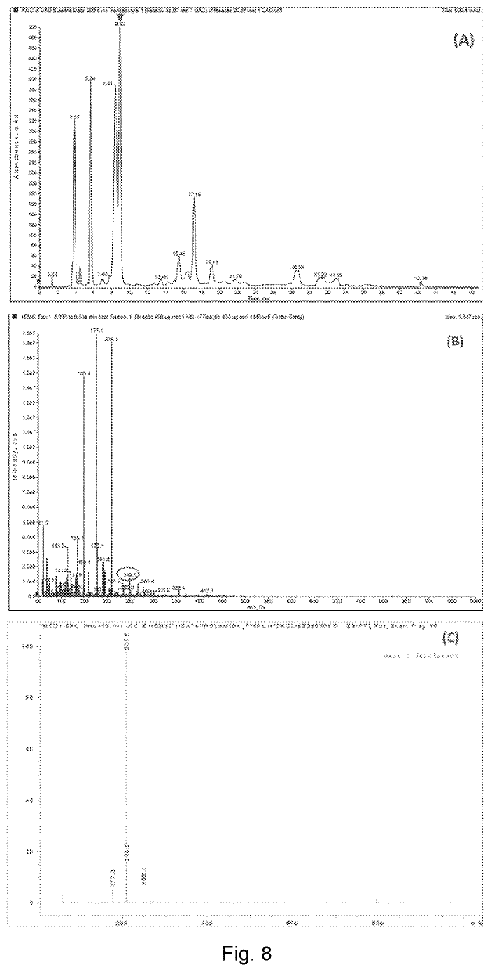

FIG. 8 shows (A): Chromatogram of the ipolamide hydrolysis product (LC-DAD, 260 nm). (B): MS spectrum of the peak with Tr=8.92 min. (C): MS spectrum of the compound 2, presented in the studies conducted by Applicant.

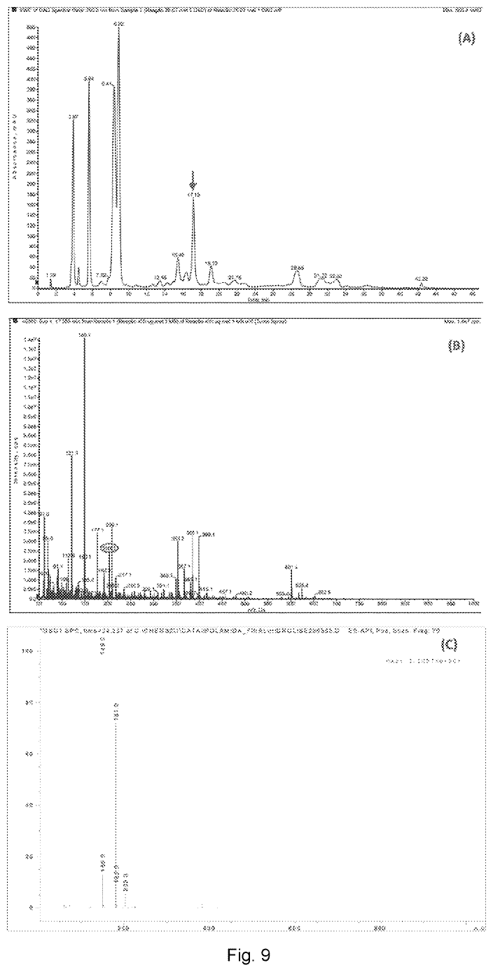

FIG. 9 shows (A): Chromatogram of the ipolamide hydrolysis product (LC-DAD, 260 nm). (B): MS spectrum of the peak with Tr=17.19 min. (C): MS spectrum of the compound 5, taken from the report sent by Applicant.

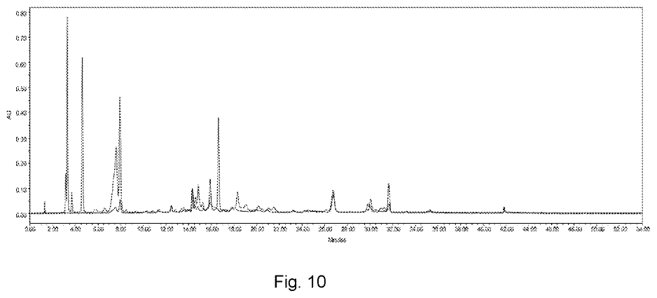

FIG. 10 shows Chromatograms (LC-DAD, 260 nm) of the reaction medium (in blue) and of the ethyl acetate fraction of the reaction medium (in black).

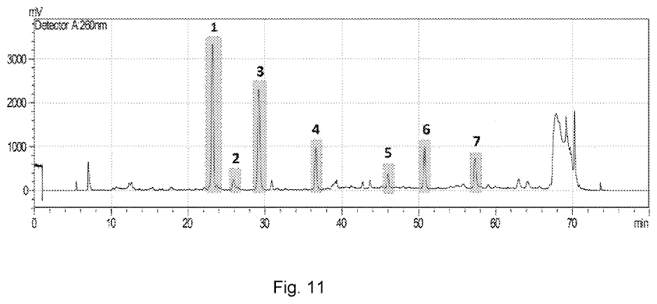

FIG. 11 shows Chromatogram (260 nm) of the ipolamide hydrolysis product subjected to LC-UV fractionation on a semi-preparative scale.

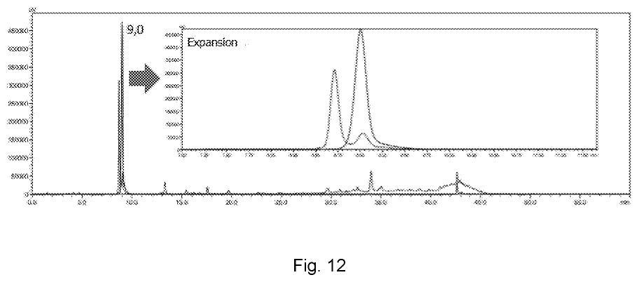

FIG. 12 shows Chromatograms (LC-DAD, 260 nm) of the ipolamide hydrolysis product (in blue) and fraction 3 (in black).

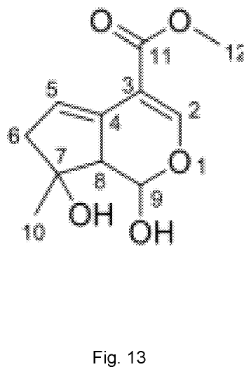

FIG. 13 shows a proposed structure for the majority constituent of fraction 3.

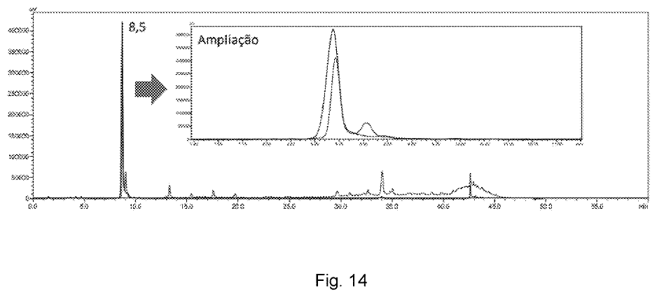

FIG. 14. Chromatograms (LC-DAD, 260 nm) of the hydrolysis product of ipolamide (in blue) and fraction 1 (in black).

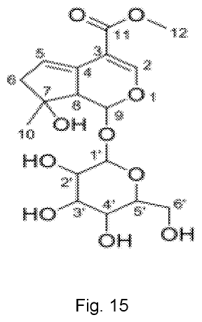

FIG. 15. Proposed structure for the majority constituent of fraction 1.

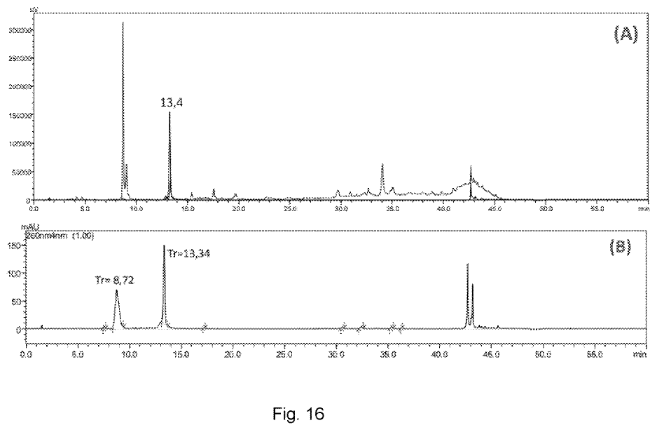

FIG. 16. (A): Chromatograms (LC-DAD, 260 nm) of the ipolamide hydrolysis product (in blue) and fraction 4 (in black). (B): Chromatogram (LC-DAD, 260 nm) of fraction 4 after 10 days of storage in the refrigerator.

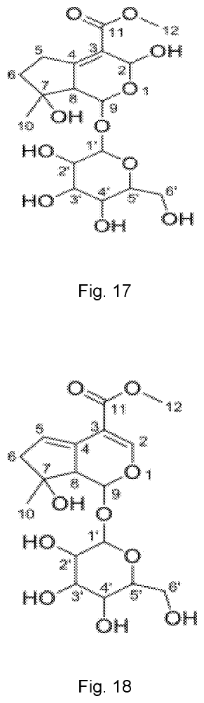

FIG. 17. Proposed structure for substance 4.

FIG. 18. Proposed structure for substance 1.

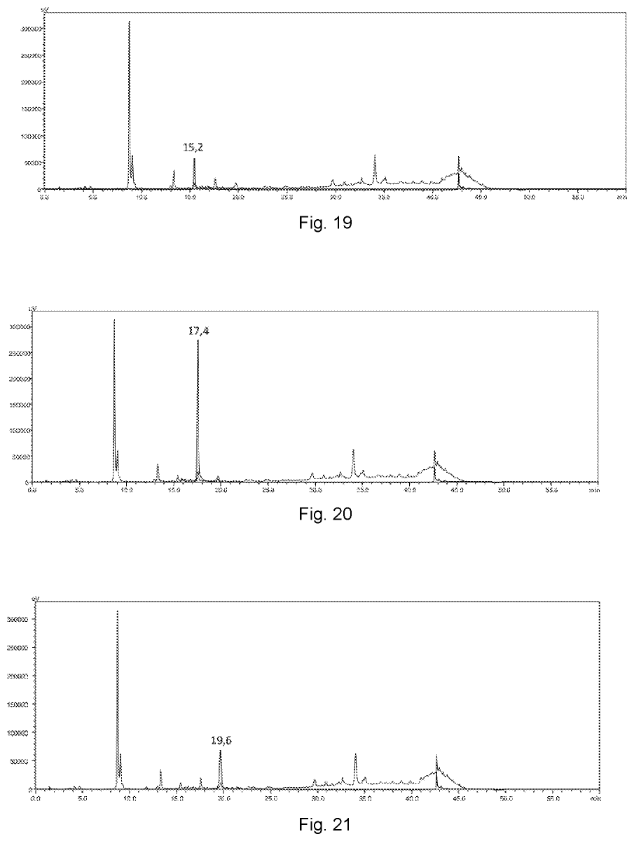

FIG. 19. Chromatograms (LC-DAD, 260 nm) of the ipolamide hydrolysis product (in blue) and fraction 5 (in black).

FIG. 20. Chromatograms (LC-DAD, 260 nm) of the ipolamide hydrolysis product (in blue) and fraction 6 (in black).

FIG. 21. Chromatograms (LC-DAD, 260 nm) of the ipolamide hydrolysis product (in blue) and fraction 7 (in black).

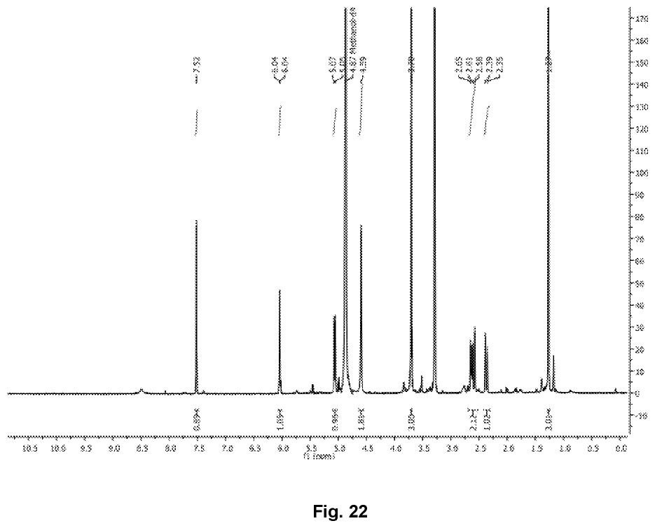

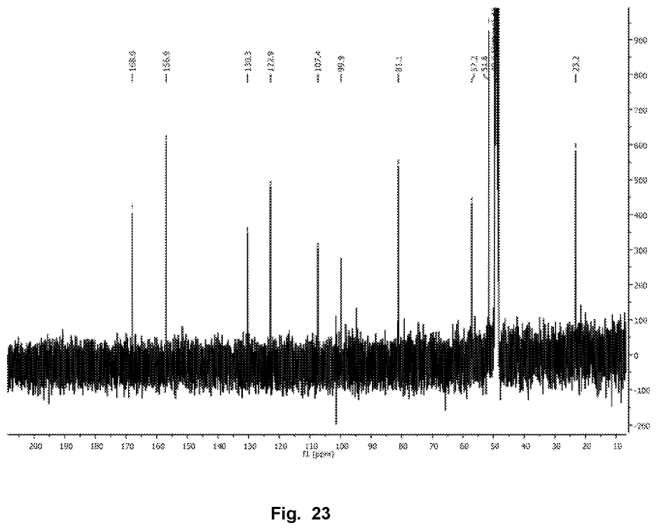

FIG. 22 shows an ¹H NMR spectrum of fraction 3 (400 MHz, CH₃OD).

FIG. 23 shows a ¹³C NMR spectrum of fraction 3 (125 MHz, CH₃OD).

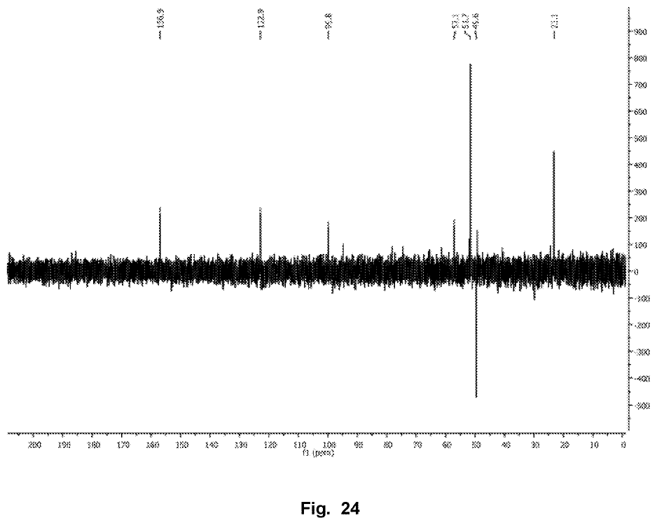

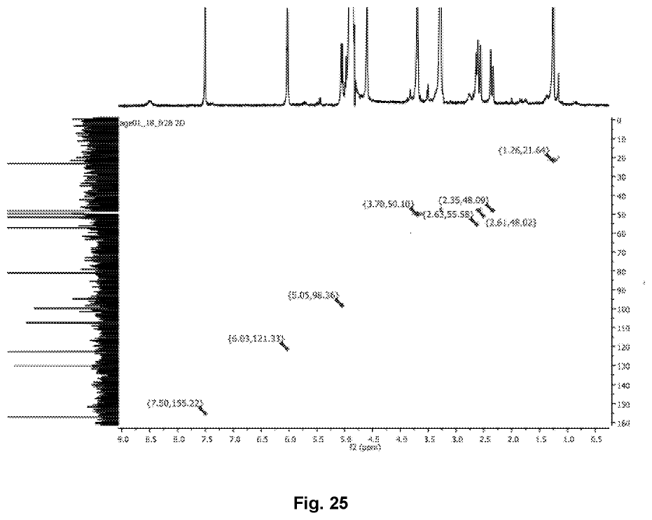

FIG. 24 shows a 135° DEPT spectrum of fraction 3 (125 MHz, CD₃OD).

FIG. 25 shows an HSQC contour map of fraction 3 (125 MHz, CD₃OD).

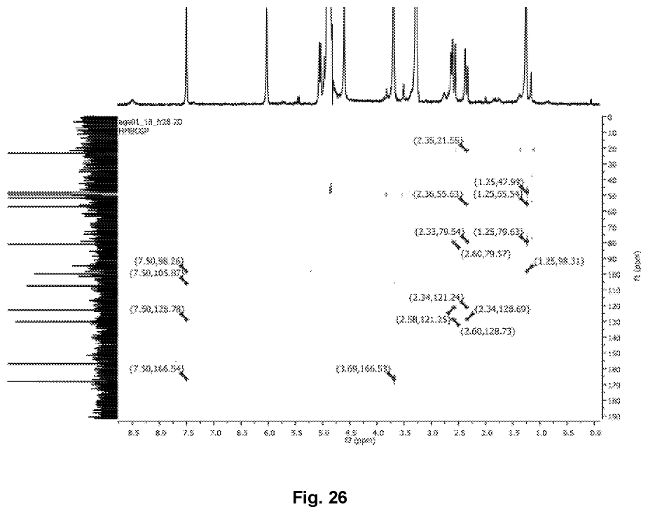

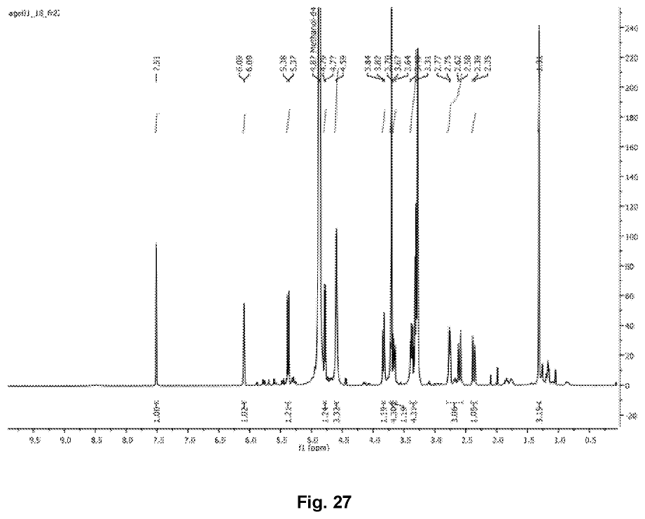

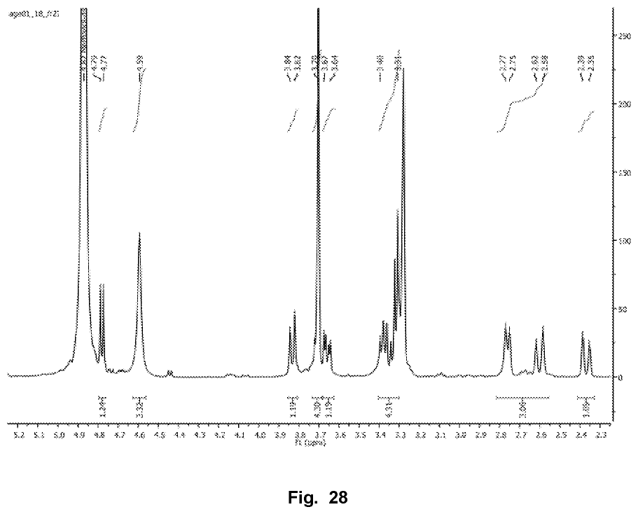

FIG. 26 shows an HSQC spectrum of fraction 3 (125 MHz, CD₃OD).

FIG. 27 shows an ¹H NMR spectrum of fraction 1 (400 MHz, CD₃OD).

FIG. 28 shows enlargement of the ¹H NMR spectrum of fraction 1 (400 MHz, CD₃OD).

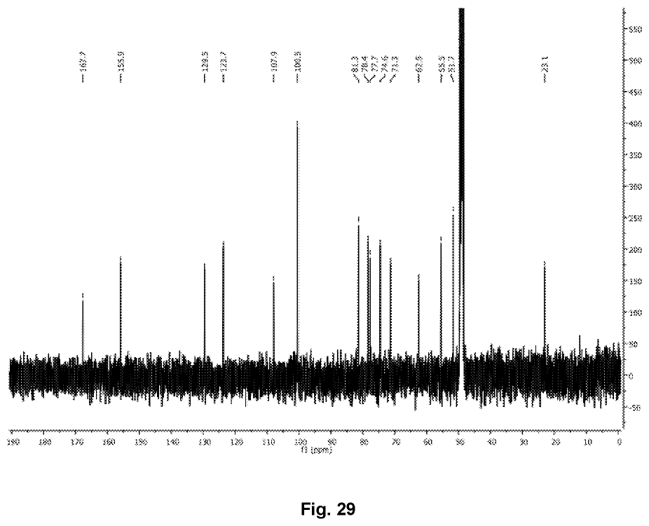

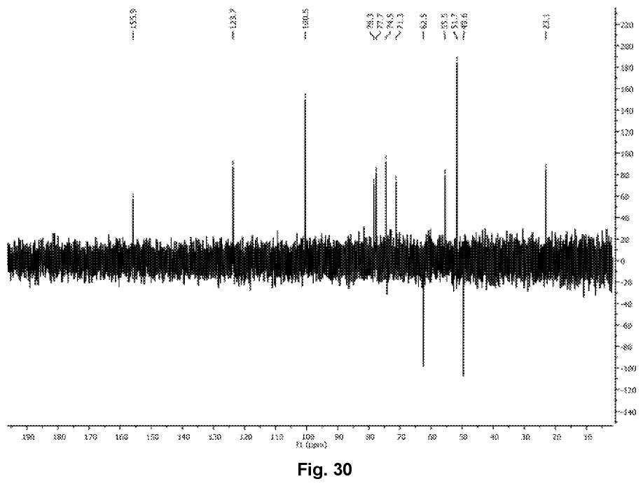

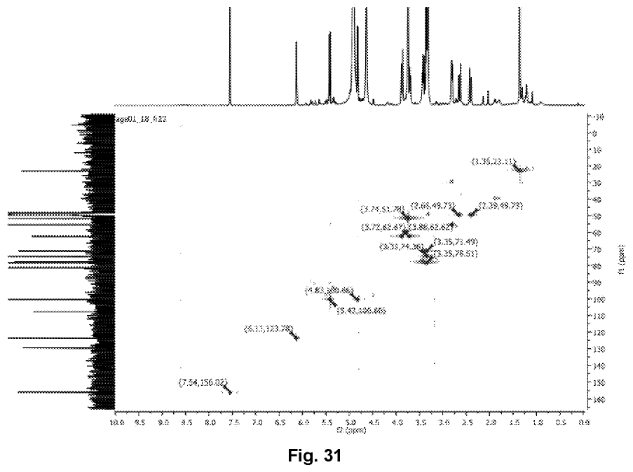

FIG. 29 shows a ¹³C NMR spectrum of fraction 1 (100 MHz, CD₃OD).

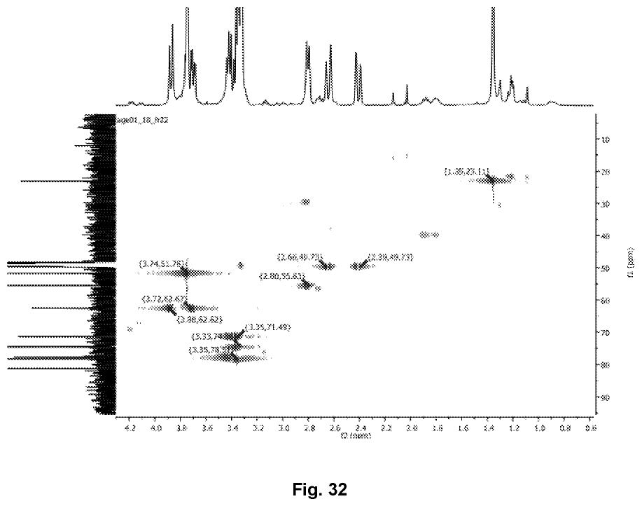

FIG. 30 shows a ¹³C NMR spectrum of fraction 1 (100 MHz, CD₃OD).

FIG. 31 shows an HSQC contour map of fraction 1 (125 MHz, CD₃OD).

FIG. 32 shows enlargement of the HSQC contour map of fraction 1 (125 MHz, CD₃OD).

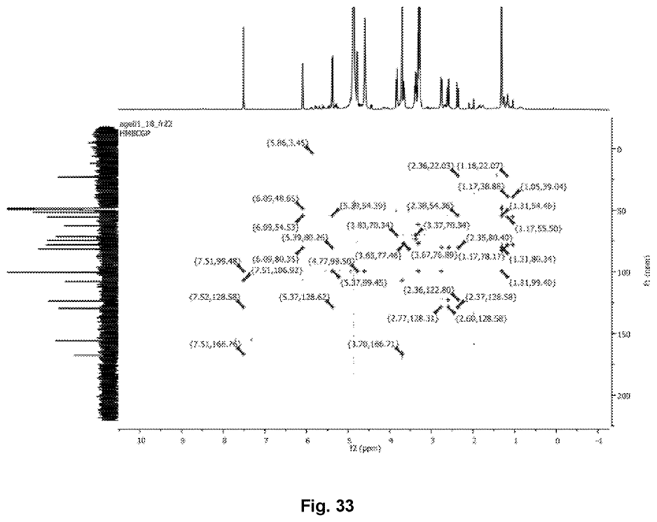

FIG. 33 shows an HMBC contour map of fraction 1 (125 MHz, CD₃OD).

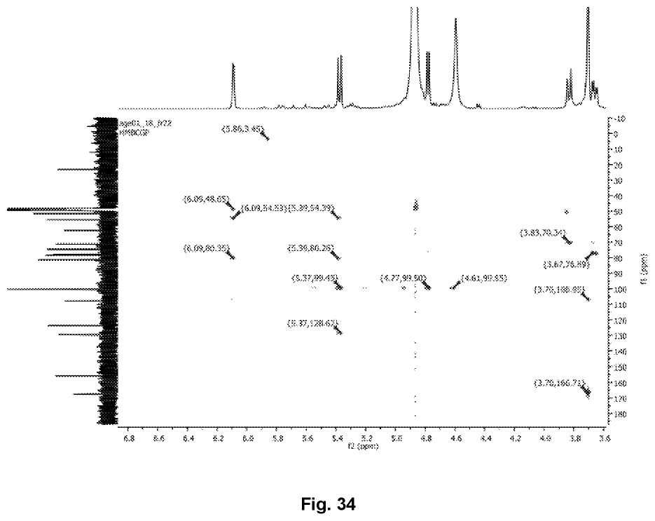

FIG. 34 shows magnification 1 of the HMBC contour map of fraction 1 (125 MHz, CD₃OD).

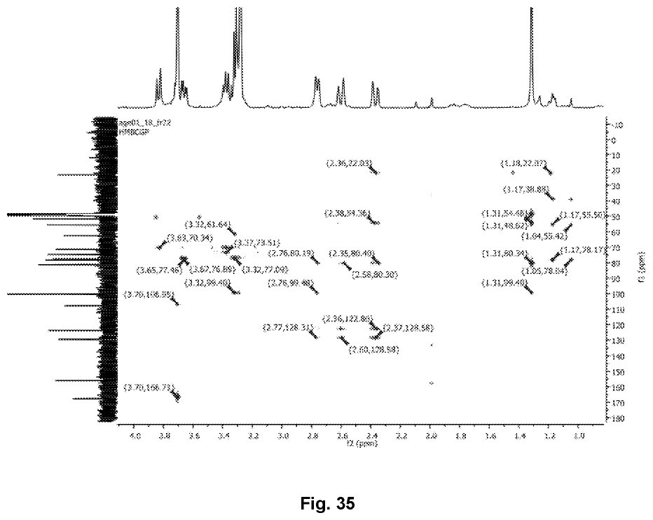

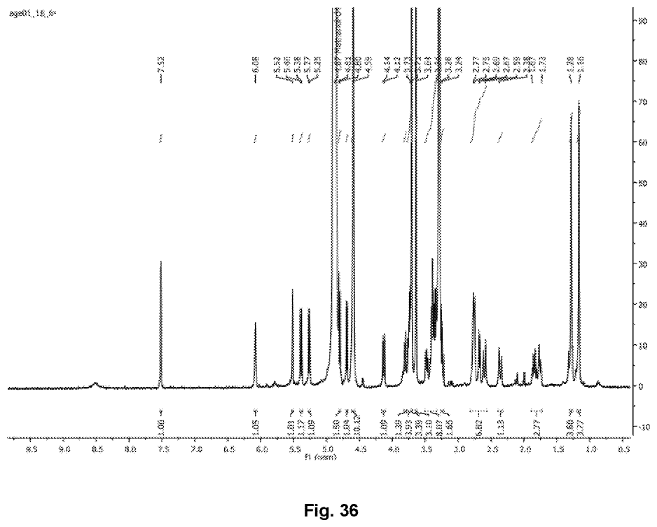

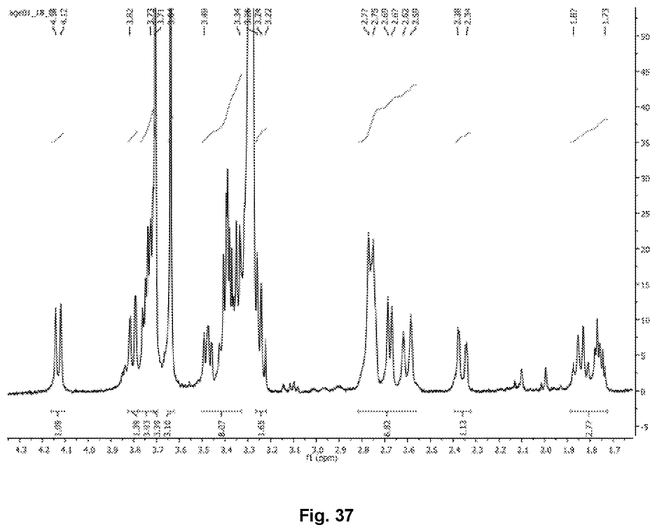

FIG. 35 shows magnification 2 of the HMBC contour map of fraction 1 (125 MHz, CD₃OD).

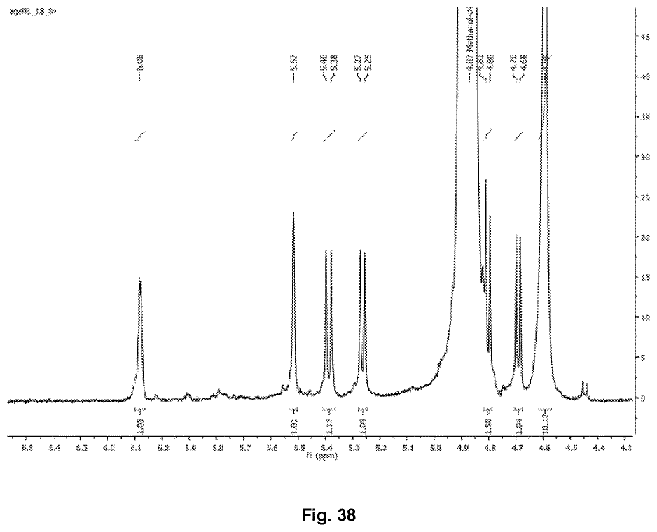

FIG. 36 shows an ¹H NMR spectrum of fraction 4 (500 MHz, CD₃OD).

FIG. 37 shows magnification 1 of the ¹H NMR spectrum of fraction 4 (500 MHz, CD₃OD).

FIG. 38 shows expansion 2 of the ¹H NMR spectrum of fraction 4 (500 MHz, CD₃OD).

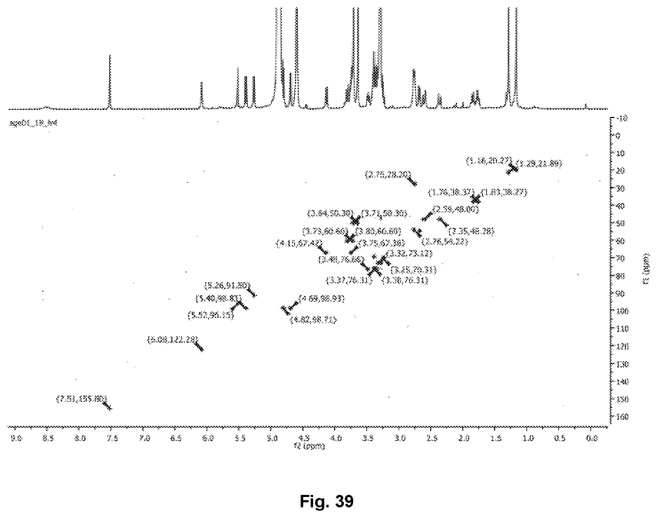

FIG. 39 shows an HMQC contour map of fraction 4 (125 MHz, CD₃OD).

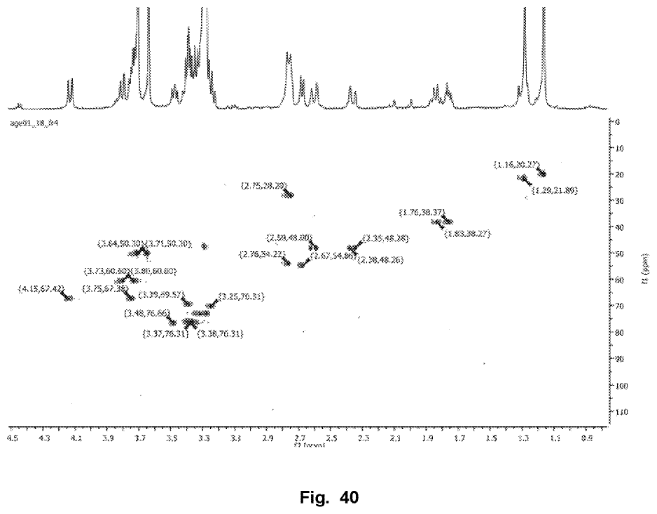

FIG. 40 shows expansion of the HMQC contour map of fraction 4 (125 MHz, CD₃OD).

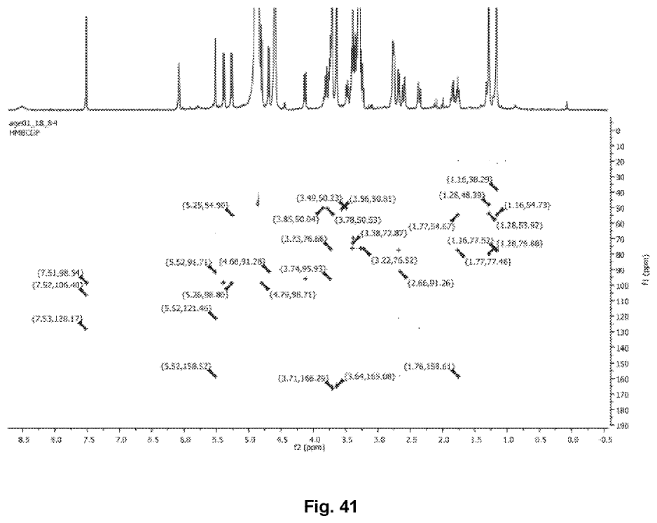

FIG. 41 shows expansion of the HMBC contour map of fraction 4 (125 MHz, CD₃OD).

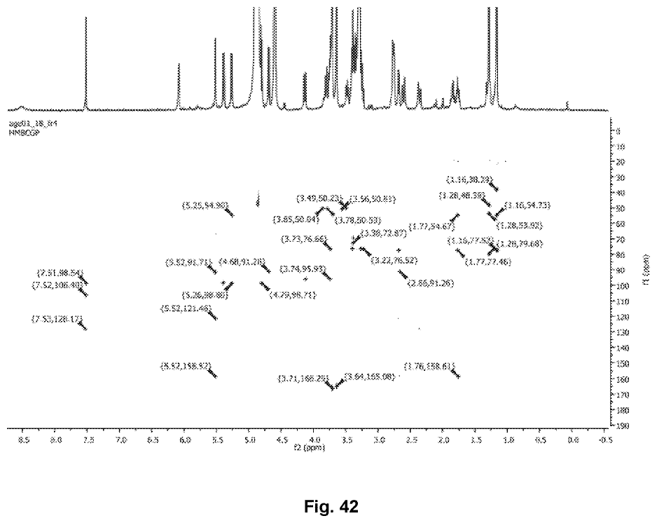

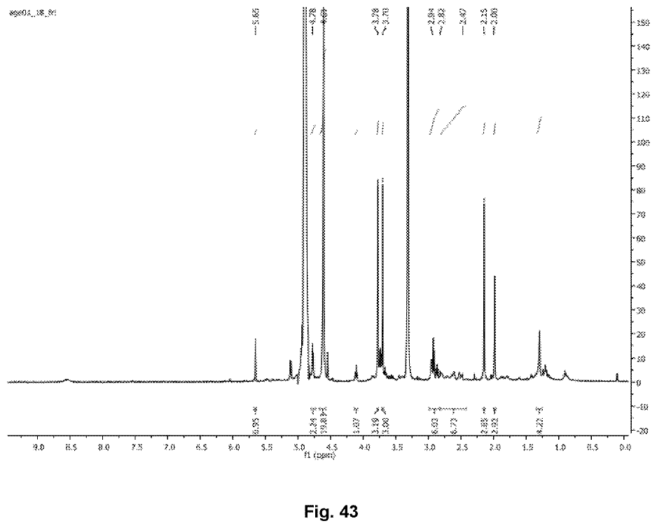

FIG. 42 shows an HMBC contour map of fraction 4 (125 MHz, CD₃OD).

FIG. 43 shows an ¹H NMR spectrum of fraction 5 (500 MHz, CD₃OD).

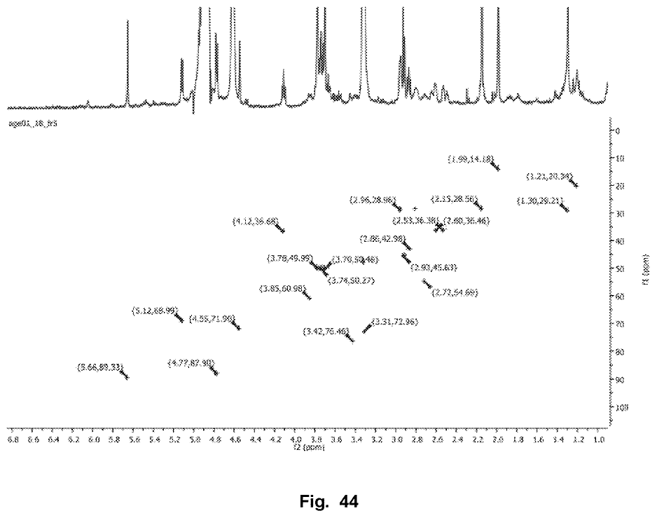

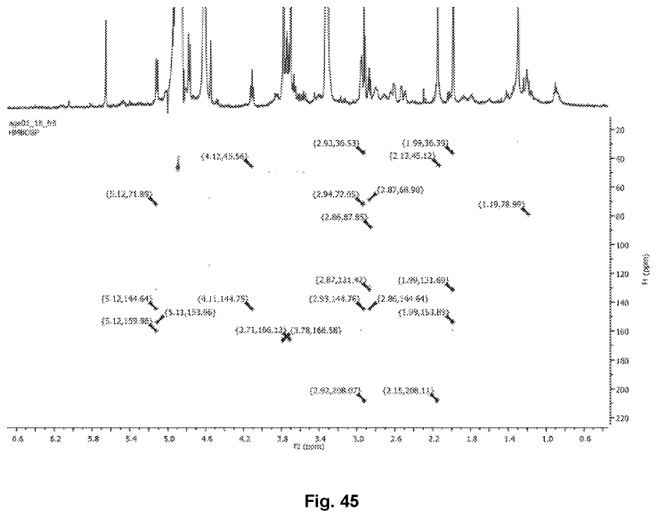

FIG. 44 shows an HMQC contour map of fraction 5 (500 MHz, CD₃OD).

FIG. 45 shows an HMBC contour map of fraction 5 (500 MHz, CD₃OD).

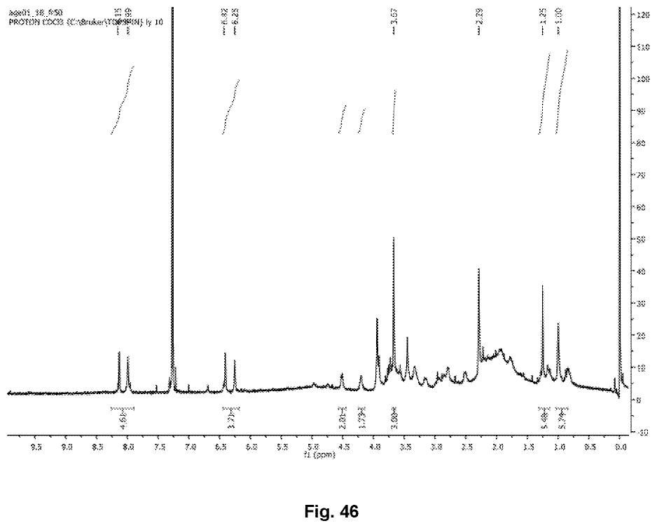

FIG. 46 shows an ¹H NMR spectrum of fraction 6 (400 MHz, CDCl₃).

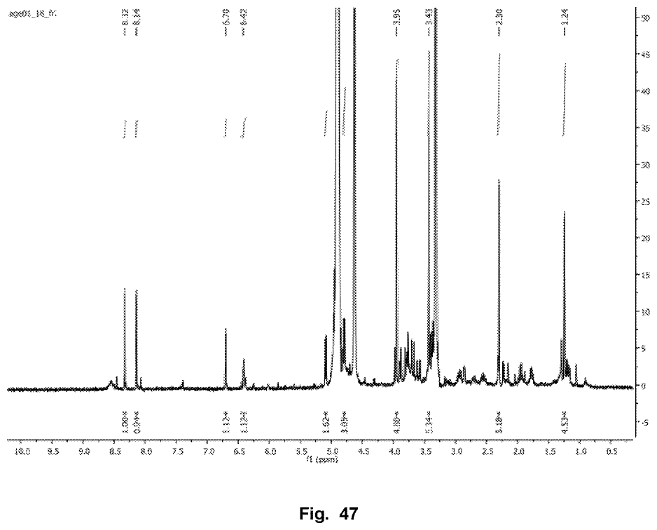

FIG. 47 shows an ¹H NMR spectrum of fraction 7 (400 MHz, CD₃OD).

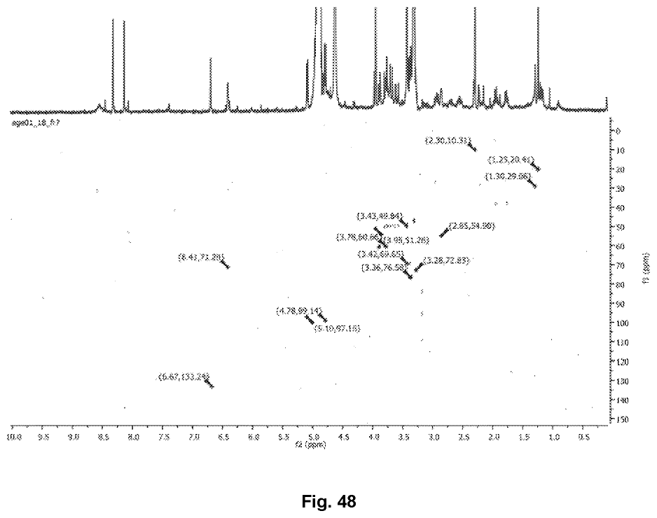

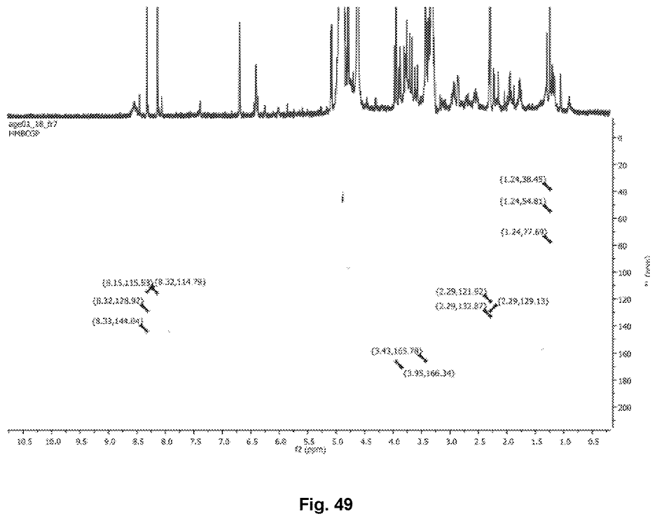

FIG. 48 shows an HMQC contour map of fraction 7 (500 MHz, CD₃OD).

FIG. 49 shows an HMBC contour map of fraction 7 (500 MHz, CD₃OD).

### DETAILED DESCRIPTION OF THE INVENTION

The examples shown herein are for the sole purpose of exemplifying one of several ways of carrying out the invention, however, without limiting the scope thereof.

### Active Compounds

The present invention presents novel and inventive compound groups, comprising the following general formulas:

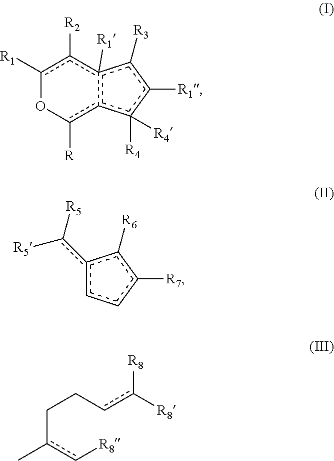

wherein R corresponds to H, OH, OGlyc (Glycoside); R₁, R_{1′}, R_{1″} correspond to H, OH; R₂ corresponds to H, COOH, COOCH₃, CH₃, CHO; R₃ corresponds to H, OH, CH₃; R₄, R_{4′} correspond to H, OH, CH₂OH, CH₃; R₅, R_{5′} correspond to H, CH₃, COOCH₃, CHO, CH₂OH; Re corresponds to CHO, COOH, COOCH₃; R₇ corresponds to H, CH₃; R₈, R_{′}, R_{8″} correspond to CHO, CH₃, CH₂OH, COOH and the dashed bonds represent single (C—C) or double (C═C) bonds between carbons (up to two double bonds per structure). In addition, R₁ may be absent or correspond to H or OH.

### Example 1: The Compounds of General Formula (I) Comprise the Following Structures

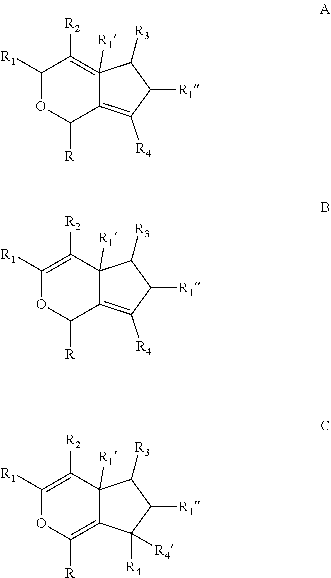

### Example 2: The Compounds of General Formula (II) Comprise the Following Structures

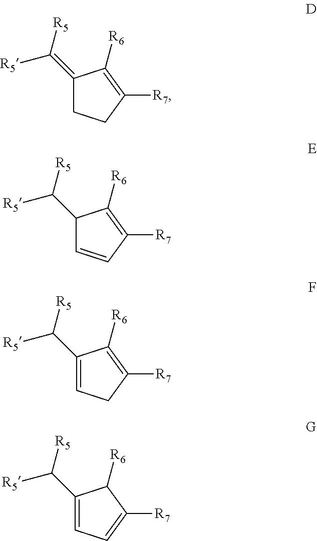

### Example 3: The Compounds of General Formula (III) Comprise the Following Structures

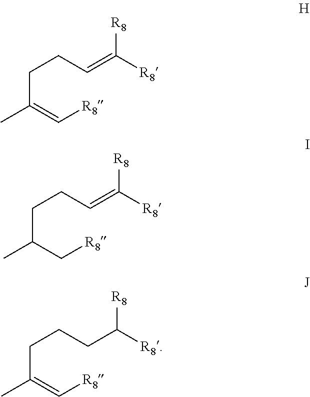

Those above mentioned structures exemplify chemical structures backbones included in formulas (I), (II) or (III).

### Immunological Disorders

The term “immune disorders” of the present invention comprises any dysfunction of the immune system. Commonly the disorders can be characterized by the components of the immune system that are affected or by the level of activity of the immune system. In the present invention, preferably, the immune disorders refer to diseases that have some evidence of autoimmunity. We can consider in the present invention vitiligo as being even more preferably chosen among the possible immune disorders.

### Method of Treatment of Immune Disorders

The present invention describes a method of treatment of immune disorders, comprising administering to a patient a compound of general formula (I), (II) and/or (III), in sufficient amount to provide immunosuppressive effect. It should be noted that, for the purpose of the present patent application, the treatment of immune disorders can be achieved using ipolamiide derivatives and/or fractions and/or extracts containing ipolamiide derivatives, any of these having immunosuppressive activity. In a preferred embodiment, the method of treatment is intended for the treatment of vitiligo.

Pharmaceutical Composition Comprising Isolated Compounds Derived from Ipolamiide.

In one embodiment, the pharmaceutical composition of the present invention comprises isolated ipolamiide derivatives, used alone or in combination, for the treatment of immune disorders, wherein the compounds comprise at least one compound selected from the group comprising:

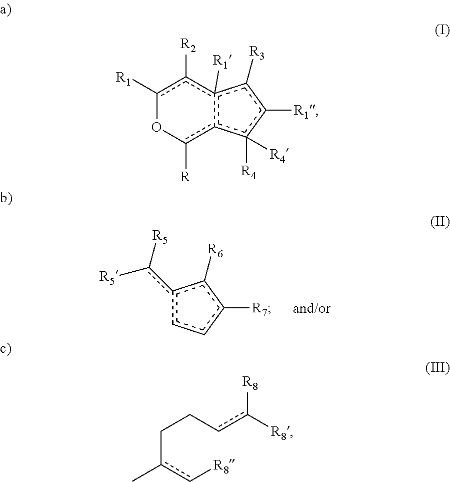

wherein R corresponds to H, OH, OGlyc (Glycoside); R₁, R_{1′}, R_{1″} correspond to H, OH; R₂ corresponds to H, COOH, COOCH₃, CH₃, CHO; R₃ corresponds to H, OH, CH₃; R₄, R_{4′} correspond to H, OH, CH₂OH, CH₃; R₅, R_{5′} correspond to H, CH₃, COOCH₃, CHO, CH₂OH; R₆ corresponds to CHO, COOH, COOCH₃; R₇ corresponds to H, CH₃; R₈, R_{8′}, R_{8″} correspond to CHO, CH₃, CH₂OH, COOH and the dashed bonds represent single (C—C) or double (C═C) bonds between carbons (up to two double bonds per structure); and
d) pharmaceutically acceptable vehicle.

In an optional embodiment, the above pharmaceutical composition further comprises the ipolamiide compound.

In a preferred embodiment, the pharmaceutical composition of the present invention comprises the compounds:

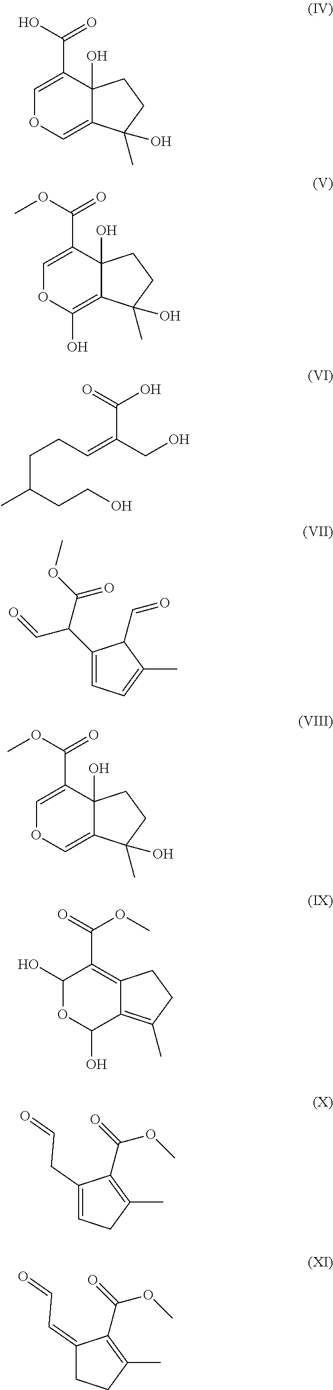

In this preferred embodiment, the compound of general formula (I) comprises the compounds of formula (IV), (V), (VIII) and (IX), the compound of general formula (II) comprises the compound of formula (VII), (X) and (XI) and the compound of general formula (III) comprises the compound of formula (VI).

The pharmaceutical composition of the present invention may additionally comprise the following compounds:

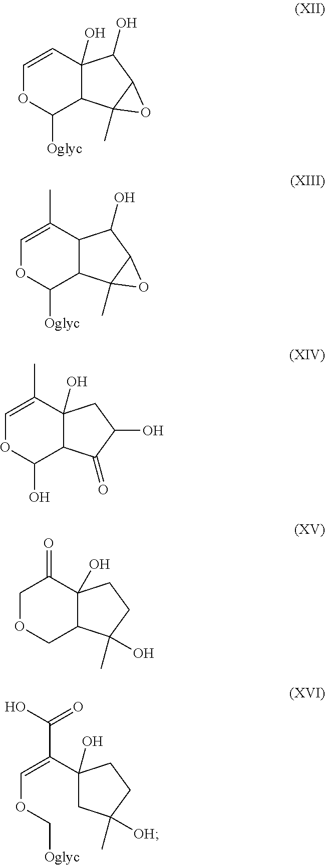

and pharmaceutically acceptable vehicle.

### Pharmaceutically Acceptable Vehicle.

To carry out their activity, the compounds of general formula (I), (II) and/or (III) should be administered to an animal organism, a mammal, particularly a human, preferably in the form of a pharmaceutical composition, i.e., associated with pharmaceutically acceptable vehicles which are suitable for each route of administration.

The pharmaceutical compositions of the present invention contain as active ingredient one or more compounds proposed herein, associated with one or more pharmaceutically acceptable vehicles. The active ingredient is commonly mixed, diluted or encapsulated with at least one vehicle.

When the vehicle is a diluent, it may be in the solid, semi-solid or liquid form, acting as a carrier, excipient or medium for the active ingredient. Therefore, the composition can be in the form of tablets, pills, powders, sachets, suspensions, emulsions, solutions, aerosols (in solid or liquid medium), creams, hard or soft capsules, suppositories, injectable solutions.

In the present invention, it is preferably considered as pharmaceutically suitable vehicle any substance different from the compound of general formula (I), (II) or (III), which has been intentionally added thereto to produce a pharmaceutical dosage form appropriate to a route of administration. Non-limiting examples of pharmaceutical excipients suitable for the preparation of pharmaceutical compositions are described in Handbook of Pharmaceutical Manufacturing Formulations—Vol. 1 to 6—2004—Sarfaraz K. Niazi—CRC Press e Remington's Pharmaceutical Sciences, Mack Publishing.

Non-limiting examples of routes of administration of the composition comprising the compound of general formula (I), (II) or (III) are the topical, oral, parenteral, nasal, rectal, transmucosal, transdermal routes.

The therapeutic dose to be employed of the compounds of the present invention should be planned and calculated according to the route of administration chosen, the age, the weight and condition of the patient and the severity of the treated disorder. In general, the compounds of the present invention are administered in therapeutically effective doses. Effective doses can be extrapolated from dose-response curves, derived from in vitro or animal models. Typically, the clinician will administer the compound until an appropriate dose to achieve the desired effect.

### Process of Production of Active Compounds

The present invention describes in detail the process of production of active compounds of general formula (I), (II) and/or (III). Essentially, the process of production of isolated compounds comprises the step of subjecting at least one ipolamiide compound to at least one heating step at high temperatures, in suitable solvent, for a sufficient time to obtain the compounds derived from ipolamiide of the general formula (I), (II) and/or (III).

In an optional embodiment, the composition of the present invention further comprises the ipolamiide compound.

In a preferred embodiment, the solvent of the present invention comprises water.

Additionally, the present invention describes the process of production of the compounds of general formula (I), (II) and/or (III), comprising the step of subjecting at least one ipolamiide compound to a heating step at high temperatures, in suitable solvent, for a sufficient time to obtain the compounds derived from ipolamiide of the general formula (I), (II) and/or (III).

In a preferred embodiment, high concentrations of the compounds of formula (I), (II) and/or (III) of the present invention are obtained from the total conversion (100%) of the content of ipolamiide used in the respective production process.

Even more preferably, the high concentrations of the present invention comprise about 0.5% to about 45% of each compound (IV to VIII) in a mixture. In this case, the concentrations comprise about 1 to about 5% of the compound IV, preferably 4%; about 15 to about 25% of the compound V, preferably 19%; about 1% to about 6% of the compound VI, preferably 3%; about 35% to about 45% of the compound VII or its isomers (e.g. compound X or compound XI), preferably 37%; about 0.5% to about 4% of the compound VIII or its isomers (e.g. compound IX), preferably 2%.

In a preferred embodiment, the high temperatures of the present invention comprise temperatures above 35° C., more preferably between 35° C. and 165° C.

In another preferred embodiment, the solvent of the present invention comprises other suitable solvents.

In a preferred embodiment, the process of production of the active compounds derived from ipolamiide comprises the step of hydrolysis or solvolysis of ipolamiide.

Even more preferably, the hydrolysis of ipolamiide may be of the acid type. Among the acids suitable for acid hydrolysis of ipolamiide, hydrochloric acid, hydrochloric, sulfuric, nitric, phosphoric and acetic acid can be mentioned, without any specific restrictions to any of them. In a preferred embodiment, in the present invention hydrochloric acid is used.

In an optional embodiment, the hydrolysis of ipolamiide may be of the basic/alkaline type. Among the bases suitable for basic/alkaline hydrolysis of ipolamiide, the alkali-metal hydroxides can be mentioned, without specific restrictions to any of them. In a preferred embodiment, in the present invention sodium hydroxide is used.

In an even more preferred embodiment, the hydrolysis in acidic medium is followed by a hydrolysis in basic/alkaline medium. As an example, we can mention the production process comprising a step of submitting at least one ipolamiide compound to high temperatures and 0.1 N (eq/L) of hydrochloric acid, followed by incubation for different time intervals that can range from 0 to 120 minutes. The hydrolysis is then interrupted by a process of neutralization using, preferably, sodium hydroxide 0.1 N.

In an optional embodiment, the production process of the compounds of general formula (I), (II) and/or (III) comprises a step of hydrolysis of ipolamiide in basic medium, being carried out with aqueous sodium hydroxide solution 0.1N, maintained at 40° C. for 2 hours.

Production Process of Active Fractions/Extracts Enriched with Ipolamiide Derivatives

It was verified by the inventors that certain isolated active compounds of the present invention can be obtained by techniques of molecular design and synthesis. At the same time, we have also specified the advantages of obtaining herbal medicines for the treatment of diseases, since these compound production systems allow a series of productive interactions between the components of the plant and the active compounds, often even synergistically. Thus, to additionally obtain an herbal medicament comprising such active compounds, we have developed an unique production process which allows to obtain an extract enriched with compounds of interest.

As described below, the extract production process of the present invention comprises unique steps that lead to extracts enriched with the ipolamiide derivatives with immunosuppressive activity. We verified the relevance of preselecting input vegetal biomasses containing between 2.5% and 3.5% of ipolamiide, resulting in an extract enriched with ipolamiide and compounds derived from ipolamiide from about 8.5% to about 11.5%. As previously presented, the vegetal biomass containing ipolamiide will be used as starting material for the production process of the extract. Only with the production process of the present invention it is possible to obtain an extract enriched with specific compounds derived from ipolamiide. This enriched extract, further, presents immunosuppressive activity.

Thus, the present invention provides a process to produce standardized extract enriched with ipolamiide derivatives from plants of the genus Stachytarpheta.

It is, therefore, an additional object of the present invention a process for production of extract enriched with compounds derived from ipolamiide, comprising essentially the steps of:
a) selecting input vegetal biomass with a content of ipolamiide between 2.5% and 3.5% obtained from plants of the genus Stachytarpheta;
b) submitting the selected biomass from a) to oven drying at temperature between 40 to 80° C., until obtaining the humidity stabilization between 10 to 12%;
c) milling the vegetal biomass;
d) performing the extraction of the vegetal biomass through the steps of:
i. heating of the vegetal biomass at a temperature between 70 to 100° C., with constant stirring;
ii. maceration of the vegetal biomass at room temperature;
iii. heating of the vegetal biomass with temperature between 70 to 100° C.

In a preferred embodiment, the process for production of the present invention further comprises the steps of:
iv. filtering and concentration of the extract;
v. drying in Spray Dryer, during 1 to 60 seconds, with inlet temperature between 155 and 165° C. and outlet temperature between 85 to 95° C., coupled to a dehumidifier.

In a preferred embodiment, the process for extraction of the present invention is an aqueous or hydroalcoholic process, even more preferably an aqueous process.

Therefore, the process for production of the present invention allows to obtain a standardized extract enriched with compounds derived from ipolamiide, preferably with a yield of about 8% to about 10%.

It is, therefore, an additional object of the present invention the extract enriched with compounds derived from ipolamiide obtained by the above-mentioned procedure. The standardized extract enriched with compounds derived from ipolamiide of the present invention comprises, preferably, the compounds of formula (I), (II) and/or (III).

The vegetal biomass of the present invention comprises all parts of plants of the genus Stachytarpheta. Preferably, the vegetal biomass comprises the aerial parts of the plants, more preferably, the leaves.

In a preferred embodiment, the input vegetal biomass comprises at least one vegetal biomass with uniform content of ipolamiide between 2.5% and 3.5%. In an optional embodiment, the input vegetal biomass comprises more than one vegetal biomass, wherein the different vegetal biomasses have different contents of ipolamiide independently, but together achieve an uniform content of ipolamiide (between 2.5% and 3.5%).

In another preferred embodiment, the actual content of ipolamiide in the input vegetal biomass can be used as a parameter for predicting the theoretical content of ipolamiide and derivatives in the extract obtained. This prediction can be accomplished by a method comprising the step of applying Equation I to some parameters obtained experimentally to find the ideal proportions of ipolamiide in the input vegetal biomass, which preferably projects the content of ipolamiide and derivatives in the extract from 8.5% to 11.5% of. The Equation I is defined below:

$$Equation I$$

$$\% Theoretical content of ipolamiide and derivatives in the extract = \frac{\begin{matrix}\% Actual content of ipolamiide \\ in the vegetal biomass \times DER\end{matrix}}{\frac{\begin{matrix}actual content of ipolamiide \\ and derivatives in the extract\end{matrix}}{\begin{matrix}actual content of ipolamiide \\ in the input vegetal biomass\end{matrix}}} \pm standard deviation$$

In this way, it is possible to predict the theoretical content of ipolamiide and derivatives in the extract from the actual content of ipolamiide in the input vegetal biomass. Preferably, the ratio between the actual content of ipolamiide and derivatives in the extract/content of ipolamiide in the input vegetal biomass is between about 3.0 and about 3.5.

The present invention further claims standardized extracts enriched with compounds derived from ipolamiide from plants of the genus Stachytarpheta obtained from the production process described above.

In an embodiment even more preferred, the plants of the present invention comprise Stachytarpheta cayennensis.

The standardized extracts from plants of the genus Stachytarpheta are preferably obtained by the production process described above resulting in an extract enriched with ipolamiide and compounds derived from ipolamiide from about 1% to about 20%, preferably from about 8.5% to about 11.5% of content of ipolamiide and derivatives.

It is, therefore, an additional object of the present invention the use of standardized extracts of plants of the genus Stachytarpheta containing compounds derived from ipolamiide for the manufacture of a medicament with immunosuppressive activity. More specifically, standardized extracts of plants of the genus Stachytarpheta containing compounds derived from ipolamiide of general formula (I), (II) and/or (III) for the manufacture of a medicament with immunosuppressive activity.

It is, therefore, an additional object of the present invention at least one active fraction of extract enriched with compounds derived from ipolamiide. Preferably, at least one fraction comprises at least one compound derived from ipolamiide of formula (I), (II) and/or (III).

In an optional embodiment, the active fraction of enriched extract further comprises ipolamiide.

It is, therefore, an additional object of the present invention the use of at least one standardized fraction enriched with compounds derived from ipolamiide, obtained from plants of the genus Stachytarpheta for the manufacture of medicament with immunosuppressive activity.

### Example 4—Additional Embodiment

The examples described in the experimental part have the sole purpose of exemplifying one of several ways of carrying out the invention, however, without limiting the scope thereof.

### Process of Production and Identification of Active Compounds

The isolated compounds of the present invention are obtained by subjecting the ipolamide compound to 0.1 N hydrochloric acid, at 40° C. and 100° C., for 1 h, 2 h and 5 h. FIG. 3 illustrates the condition at 40° C. From this experiment, we could observe several products from this hydrolysis and, based on chromatograms, identify several derivatives of ipolamiide, such as, for example, the structures described below, as illustrated on FIG. 3.

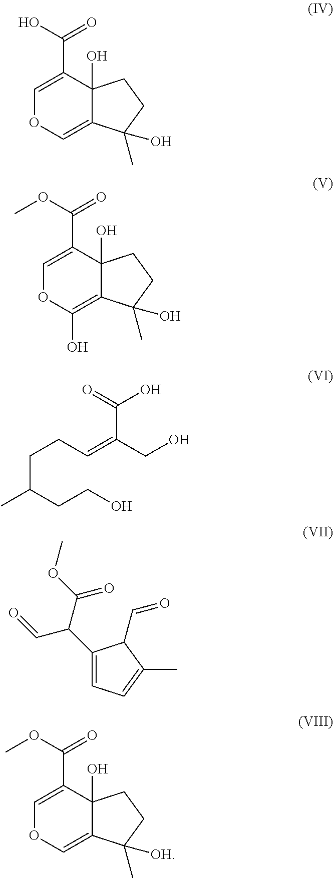

Alternatively, the hydrolysis can be carried out by varying the hydrochloric acid concentration between 0.1 to 1 N and the experimental temperature may vary between 35° C. and 165° C. In addition, the hydrolysis time can vary between 1 minute and 24 hours to facilitate the formation of higher concentrations of certain ipolamiide derivatives.

### Process for Obtaining the Extract

According to the present invention, the process for obtaining aqueous extract of Stachytarpheta cayennensis rich in ipolamiide derivatives mainly comprised the steps of producing a standardized extract illustrated in the flowchart of FIG. 1 to obtain material for the development of pre-clinical research in immunology.

At first, seeds preferably selected by genotyping of Stachytarpheta cayennensis were submitted to a process of seeding for two months in a controlled environment regarding temperature, humidity and light. The seeding was carried out in expanded polystyrene trays, filled with substrate, and kept in this protected environment with controlled irrigation. The seedlings began to appear in 10 to 15 days. The trays remained in these conditions until the seedlings reached size and ideal conditions for permanent transplantation.

The seedlings with an approximate size of 5 to 8 cm in height and with 2 to 3 pairs of definitive leaves were transplanted to the growing site, which preferably had an annual average temperature of 30° C., annual average relative humidity of less than 55%, and in which the soil had preferably, but not limited to, the results of specific chemical and physical soil analyses, for example, acidity, calcium, nitrogen and use of organic fertilization in all areas.

After planting the seedlings, the first harvest was carried out after 6 months, and the other regrowth every 4 months, thus guaranteeing an optimized life cycle for the shrub aiming at maximizing ipolamiide content in the input vegetal biomass.

Following the planting, the vegetal biomass was stabilized through a greenhouse drying process, under defined conditions of temperature and humidity. The plants were dried in dryers with heat exchanger, forced air circulation, and temperature ranging from 50 to 70° C., preferably 60° C. The drying consisted of the passage of hot air through the plants, removing the humidity, until the vegetal biomass was stabilized with humidity between 10 to 12%. In a preferred embodiment, the drying time occurs from 8 to 20 hours.

As shown in FIG. 1, the vegetal biomass was subjected to the milling process by means of a hammer mill with 1800 RPM and a 19 mm sieve, obtaining a productivity of 50 to 150 kg/hour at room temperature. After milling, the vegetal biomass was submitted to an aqueous extraction at temperature between 80 to 90° C., with constant stirring for 15 min. The amount of extractive solution used should be 10× the amount of vegetal biomass used, guaranteeing exhaustive extraction of the substance of interest in the vegetal biomass.

After the previous step, the material was submitted to the maceration process for 10 h at room temperature. Subsequently, the material was again heated at 80 to 90° C. for 15 min.

The material was filtered on a rotary filter with polyester mesh of 40 μm. After the filtration step, the material was concentrated on a “Bernauer” evaporator and/or falling film evaporator to about 30% of total solids.

The product of this step was submitted to drying in Spray Dryer, with inlet temperature between 155 to 165° C. and outlet temperature between 85 to 95° C. coupled to a dehumidifier, preferably of the Bry-Air type, during 20 to 40 seconds, aiming at obtaining the lowest residual humidity content possible (Table 1). This process substantially improves the quality of the enriched extract, since the residual humidity in the material initially compromises the stability of the components of interest, during the shelf life of the vegetal extract/derivative. This extractive process resulted in a ratio of 10 to 12:1 and yield varying between 8 to 10%.

TABLE 1
Humidity content in the extract after drying
only and after drying with humidifier

| Humidity content in the | Humidity content in the extract after |
|---|---|
| extract after drying with SD | drying with SD + dehumidifier |
| 4.61% | 1.77% |

The extractive process described above guaranteed the exhaustive extraction of ipolamiide to assure the immunosuppressive activity of the extract due to the presence of ipolamiide derivatives generated in the process. Therefore, it was necessary to establish process controls for the input vegetal biomass, so that the actual content of ipolamiide and derivatives in the extract was between 8.5 and 11.5%, as follows in Table 2. The control of ipolamiide in the vegetal biomass ensures the presence of ipolamiide derivatives with immunosuppressive activity in the extract, generated by the process described herein.

TABLE 2
Content of ipolamiide in the vegetal biomass and in the extract.

| Actual content of | Theoretical content | Actual content | Selection |
|---|---|---|---|
| ipolamiide in the | of ipolamiide and | of ipolamiide | of input |
| input vegetal | derivatives | and derivatives | vegetal |
| biomass | in the extract | in the extract^{a} | biomass |
| 1.57% | 5.23% | 4.47% | Not selected |
| | (4.44-6.02) | | |
| 2.20% | 7.33% | 7.62% | Not selected |
| | (6.23-8.43) | | |
| 3.00% | 10% | 10.80% | Selected |
| | (8.5-11.5) | | |
| 3.30% | 11% | 11.20% | Selected |
| | (9.35-12.65) | | |
| 3.07% | 10.2% | 8.90% | Selected |
| | (8.67-11.73) | | |
| 3.65% | 12.2% | 12.40% | Not selected |
| | (10.37-14.03) | | |

^{a}The agreement between the theoretical and actual contents of ipolamiide and its derivatives in the extract demonstrates the applicability of Equation I.

With this, it was possible to determine a method for predicting the theoretical content of ipolamiide and its derivatives in the enriched active extract. For this purpose, it was used an equation to project the content that would be obtained by the extraction process (theoretical) from the actual content of ipolamiide in the vegetal biomass (Equation I).

$$Equation I$$

$$\% Theoretical content of ipolamiide and derivatives in the extract = \frac{\begin{matrix}\% Actual content of ipolamiide \\ in the vegetal biomass \times DER\end{matrix}}{\frac{\begin{matrix}actual content of ipolamiide \\ and derivatives in the extract\end{matrix}}{\begin{matrix}actual content of ipolamiide \\ in the input vegetal biomass\end{matrix}}} \pm standard deviation$$

In the above-described equation, DER can be understood as the amount of vegetal biomass required to obtain 1 kilo of native extract. The ratio between the actual content of ipolamiide and derivatives on the extract/content of ipolamiide in the input vegetal biomass is, preferably, between about 3.0 and about 3.5.

Thus, in a prospective way, using the above equation, it is possible to select only the vegetal biomass (whose content of ipolamiide is obtained by analytical methods, such as HPLC) that projects an adequate theoretical content of ipolamiide and derivatives in the extract between 8.5 and 11.5% and discard those that will not project this range. In a novel and inventive manner, it has been verified in the present invention that the relationship between the actual content of ipolamiide and derivatives in the extract and the content of ipolamiide in the input vegetal biomass will, preferably, be between 3.0 and 3.5.

The HPLC analysis comprised the steps of preparing sample solutions and standards, and elution thereof, as follows:

### 1. Solutions Preparation

1.1—Formic acid solution 0.1% (mobile phase A): In a 1000 mL volumetric flask containing approximately 800 ml of ultra-pure water, it was added 1 ml of formic acid. The flask volume was filled with ultra-pure water and well homogenized.
1.2—Formic acid diluent solution: methanol (1:1): In a beaker, it was mixed 50 mL of the 0.1% formic acid solution with 50 mL of methanol.

### 2. Sample Preparation:

Raw-material (Herbal): 1.0 g of the ground herbal was weighed and transferred to an amber 250 ml Erlenmeyer flask or covered with aluminum foil. 50 mL of distilled water was added and extracted under reflux at 80° C. for 2 hours. The solution was paper-filtered into a 50 mL volumetric flask and filled up with distilled water. It was filtered through 0.22 OR 0.45 μm membrane to an HPLC vial.

### 3. Standards Preparation:

Ipolamiide standard 100 ppm: 1.0 mg of ipolamiide standard was weighed and transferred to an amber 10 mL volumetric flask. 5 ml of the diluent was added, which was left in an ultrasonic bath for 10 minutes or until complete dissolution. It was filled with the diluent and then there was the homogenization. Humidity analysis by Karl Fischer was performed for the ipolamiide standard.

### 4. Analysis by HPLC:

### 4.1—Parameters/Equipment

Column: Zorbax SB-C18 (250 mm×4 mm; 5 μm)
Mobile phase: (A) 0.1% formic Acid;
   (B) Methanol.

TABLE 3
Ipolamiide Elution Gradient

| Time (min) | (%) A | (%) B |
|---|---|---|
| 0 | 80 | 20 |
| 27 | 58 | 42 |
| 32 | 58 | 42 |
| 32.1 | 0 | 100 |
| 36 | 0 | 100 |
| 36.1 | 80 | 20 |
| 45 | 80 | 20 |

Flow rate: 1.0 mL/min
Detection: 254 nm.
Analysis time: 45 minutes
Injection volume: 30 μL

### 4.2—Calculation of Ipolamiide Content

$$Content of ipolamiide ( \% ) = A sample \times M standard \times P standard \times D sample \times ( 100 - U standard ) ;$$

$$A standard \times M sample \times D standard \times 10000 / 100 ;$$

wherein:
A sample: Peak area of ipolamiide in the sample
M standard: Mass of the standard ipolamiide in mg
P standard: Purity of the standard in decimal
D sample: Dilution of the sample in mL
A standard: Peak area of ipolamiide in the standard
M sample: Mass of the sample used in g
D standard: Dilution of the standard in L
U standard: Humidity of the standard quantified by Karl Fischer
10000: Conversion of units

The result of the content in the extract was given in dry base, that is, the humidity was discounted. Therefore the calculation for the mass of the extract was:

$$M sample = Mass \times ( 100 - U sample ) / 100 ;$$

wherein:
Mass=Mass of dry extract (in grams)
U sample=Humidity of the dry extract in percentage, according to iT2-052.

### 5. Analysis by HPLC for Ipolamiide Derivatives

### 5.1—Parameters/Equipment

Column: Eclipse XDB Agilent-C18 (150×4.6 MM) 5 MICRONS
Mobile phase: (A) 0.1% formic Acid buffer;
   (B) Acetonitrile

TABLE 4
Ipolamiide Derivatives Elution Gradient

| Time (min) | (%) A | (%) B |
|---|---|---|
| 0 | 95 | 5 |
| 5.00 | 90 | 10 |
| 8.00 | 80 | 20 |
| 10.00 | 90 | 10 |
| 12.00 | 95 | 5 |

Flow: 1.2 mL/min
Detection: DAD (205-280 nm).
Analysis time: 15 minutes
Injection volume: 20 UL

### 5.2 Calculation of Content for Ipolamiide Derivatives

For analysis of a given ipolamiide derivative (Y), we submitted it to a solution with defined concentration. This concentration is directly correlated with the area observed in the chromatogram. This area, when compared with the total area of the chromatogram, multiplied by the concentration previously defined, give us the percentage value of the said derivative (Y) in the sample, as can be seen below:

$$Sample concentration \times \frac{Derivative Y area}{Total area} \times 100 = \% of derivative Y content$$

$$( \sum content of derivatives = \% total content of ipolamiide derivatives in the sample . )$$

### Biological/Immunosuppressive Activity

The evaluation of the in vitro biological activity of the aqueous extract of Stachytarpheta cayennensis as well as of ipolamiide and its derivatives was conducted as described below.

The extract of Stachytarpheta cayennensis, ipolamiide and derivatives were studied in an experimental in vitro immunological model involving CD8+ T cells and IFNγ. The objective of this study was to evaluate whether the extract and other substances, isolated or mixed, act by blocking the activation of CD8+ T cells and the consequent secretion of IFNγ.

Peripheral blood mononuclear cells (PBMCs) from healthy volunteers were isolated from leukocyte layers by Ficoll-Hystopaque centrifugation. Thereafter, human CD8+ T cells were isolated using the CD8+ T cell isolation kit (Miltenyi Biotec, #130-096-495). These cells (3×10⁵ cells/well) were incubated in RPMI+10% of FBS medium, activated with αCD3/CD28 (1 μg/mL) and treated with different concentrations of aqueous extract of Stachytarpheta cayennensis standardized in 10% of ipolamiide, isolated ipolamiide, and ipolamiide derivatives obtained by acid hydrolysis to study their effect on the prevention of the CD8+ T cell activation and secretion of IFNγ. For evaluation of cell proliferation, bromodeoxyuridine (BrdU), a thymidine analogue commonly used for proliferation assays, was used as a marker for the proliferation. Specifically, it was evaluated the incorporation of BrdU through the Biotrak ELISA System (GE Healthcare, RPN250) per the manufacturer's instructions. The methodology for quantification of interferon gamma comprised the use of the Human IFNγ ELISA Ready-SET-Go kit (eBiosciences 88-7316-88) and followed the manufacturer's instructions. As illustrated in the graph set of FIG. 2, the aqueous extract of Stachytarpheta cayennensis enriched with ipolamiide derivatives blocked the activation of CD8+ T cells induced by αCD3/CD28. Isolated ipolamiide had no effect on the proliferation of CD8+ T cells induced by αCD3/CD28, or the secretion of IFNγ. However, the compounds derived from ipolamiide in a mixture obtained for its acid hydrolysis significantly reduced the CD8+ T cell proliferation and the secretion of IFNγ. The ipolamiide derivatives isolated from this mixture also demonstrated a statistically significant reduction of CD8+ T cell proliferation and of secretion of IFNγ. Tacrolimus, a well-known immunosuppressive agent, was used as a reference compound and as a positive control for the experiment. The effect obtained by tacrolimus was similar to that obtained with hydrolyzed ipolamiide in both cell proliferation and IFNγ production.

Thus, the results showed that the immunosuppressive activity derives from the ipolamiide derivatives and not from the intact molecule. It is important to reinforce that the compounds obtained by the experimental condition of acid hydrolysis of isolated ipolamiide are present in the aqueous extract of Stachytarpheta cayennensis, as shown in FIG. 4. However, the presence of such compounds is due to the extraction process used in the present invention.

### Example 5—Isolation of Compounds Formed from the Acid Hydrolysis of Ipolamide

### Summary and Conclusions

In studies conducted by the present Applicant, it was reported that the hydrolysis of ipolamide in an acidic medium leads to the formation of several products, among which are compounds 1 and 5, which chemical structures were provided to Lychnoflora. This present study had as objectives the evaluation of the feasibility of isolating 100 mg of each of these, as well as their structural conformations.

The hydrolysis product of ipolamide was analyzed by LC-DAD and LC-MS, using the chromatographic conditions described in the report sent by Applicant. From the comparison between the chromatographic profile obtained in this study with that presented in the previous study provided by Applicant, together with the m/z information relating to each chromatographic peak, the peaks that would possibly correspond to compounds 1 and 5 described in the report sent by Applicant and targets of this study were suggested.

Subsequently, different chromatographic techniques were performed to fractionate the product of the ipolamide hydrolysis, wherein the fractionation by LC-UV on semipreparative scales allowed the isolation of major products obtained from ipolamide. The fractions obtained were analyzed by LC-DAD, and their respective chromatographic purities were determined.

These main fractions were also analyzed by 1D (¹H and ¹³C) and 2D (HMQC and HMBC) NMR. None of the obtained spectra presented signals that could be attributed to the molecules identified in the report provided by Applicant. The compilation of the spectral data obtained allowed the structural suggestion of three isolated substances, indicated below, which are, in general, the result of dehydration reactions and glucose loss.

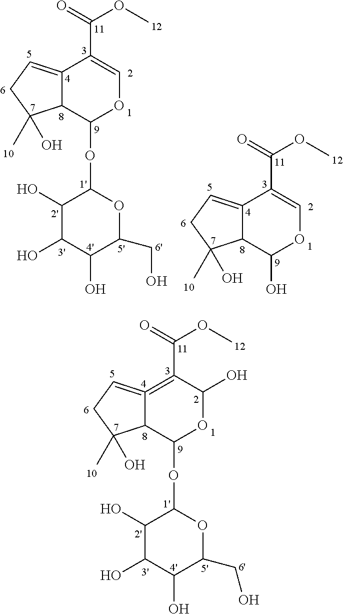

### 1 PRELIMINARY INFORMATION

In the studies referenced above, it was reported that the hydrolysis of ipolamide in acid medium leads to the formation of several products, among which are compounds 1 and 5.

In this same report, the isolation of compound 1 was described. The characterization by mass spectrometry and structural determination by NMR suggested that it was a mixture containing molecules A and B. Additionally, it was suggested by Applicant that compound 1 also could correspond to structure C as shown below:

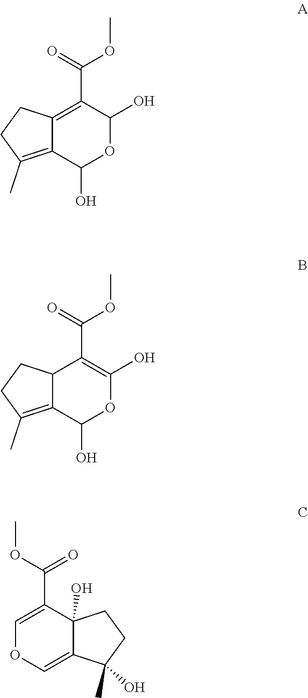 [SMILES: COC(=O)C1=C2CCC(C)=C2C(O)OC1O ; COC(=O)C1=C(O)OC(O)C2=C(C)CCC12 ; COC(=O)C1=COC=C2[C@]1(O)CC[C@]2(C)O]

It was also reported that another major product of the reaction would be compound 5. In the material sent, the isolation and characterization of this compound by mass spectrometry was reported, and it was possible to suggest the structure presented below for compound 5:

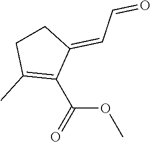

It was reported there was a need for isolation and confirmation of the characterization of compounds 1 and 5, and assessment of the feasibility of scaling up the isolation process to obtain 100 mg of these substances.

### 2 OBJECTIVES

The aim of this study was to assess the feasibility of isolation and structural confirmation of the substances formed from the acid hydrolysis of ipolamide, named as Compound 1 and Compound 5.

### 3 EXPERIMENTAL DESIGN

The flowchart of FIG. 3 shows the design used to carry out this study. Initially, acid hydrolysis reaction of ipolamide was carried out. Then, the hydrolysis product was analyzed by LC-DAD and LC-MS, and these analyses allowed us to suggest the peaks possibly corresponding to the compounds of interest.

Afterwards, the reaction was scaled up with the aim of obtaining a greater mass of hydrolyzed product for the subsequent isolation of the compounds of interest. Fractionations were carried out by flash column chromatography, using silica gel as the stationary phase and elution with chloroform:methanol. The collected fractions were grouped according to their chromatographic profiles obtained in the analysis by thin layer chromatography (TLC). Then, the grouped fractions were analyzed by LC-DAD, wherein peaks were possibly observed corresponding to compounds 1 and 5. However, the chromatograms of the fractions with the compounds of interest indicated the presence of interferences and the need to optimize chromatography conditions for isolation.

Thus, a new fractionation was performed by classical column chromatography (CCC) with silica gel and hexane:ethyl acetate as the mobile phase. The collected fractions, grouped after analysis by TLC, were analyzed by LC-DAD. It was observed that some fractions presented peaks corresponding to the compounds 1 and 5 with a higher purity degree, but still with the presence of interfering.

Given the difficulties in isolating the substances of interest and the possibility of degradation of these substances during fractionation, which was reported in the previous report provided by Applicant (elution and drying processes, for example), fractionation by LC-UV was chosen in semipreparative scale. The collected fractions were dried by lyophilization, analyzed by LC-DAD, and the isolation of substances with a satisfactory purity degree for structural identification was observed.

The fractions were analyzed by NMR, which allowed the structural determination of three derivatives formed from the degradation of the ipolamide. The experimental design of the AGE 01-18 project. is shown in FIG. 5.

### 4 EXPERIMENTAL PROCEDURES

### 4.1 Samples, Materials, Reagents and Equipment

### Samples:

Ipolamide—Stachytarpheta cayennensis extract, Applicant Batch: 1710001, Exp.: Oct. 30, 2019.

### Reagents, Organic Salts and Solvents:

Formic acid grade HPLC 88%;
Ethyl acetate PA;
Acetonitrile HPLC grade;
0.1 N hydrochloric acid solution;
0.1% formic acid solution;
0.1 N sodium hydroxide solution.

### Equipment:

LC-DAD analysis: Shimadzu UFLC liquid chromatograph, equipped with pumps model LC-20AD, DAD UV/VIS SPD-M20A detector, SIL-20 AC HT auto-injector, CTO-20A oven and CBM-20A controller coupled to Bruker Daltonics mass spectrometer, model amaZon SL, with Ion Trap analyzer;
LC-UV isolation on semipreparative scale: Shimadzu liquid chromatograph with two pumps model LC-6AD, SPD-20A detector, 2 mL loop injector, DGU 20A5 degasser, FRC-10A fraction collector and CBM-20A5 controller;
LC-DAD-MS analysis: Shimadzu UFLC liquid chromatograph, equipped with pumps model LC-20AD, DAD UV/VIS SPD-M20A detector, SIL-20 AHT auto-injector, oven CTO-20A and CBM-20A controller coupled to Bruker Daltonics mass spectrometer, model amaZon SL, with Ion Trap analyzer.
Nuclear Magnetic Resonance (NMR) Analysis: Bruker-Advance DRX 400 Spectrometer with 9.39 Tesla, operated at 400 MHz for ¹H.
Freeze-drying was carried out in a Labconco freeze-dryer, model FreeZone 2.5.

### 4.2 Acid Hydrolysis Reaction of Lipoamide

The reactions were carried out according to the procedures described in the report sent by Applicant. A 50 mg aliquot of ipolamide was solubilized in 1 mL of 0.1 N hydrochloric acid. The solution was heated to 37° C. for 2 hours and then neutralized by the addition of 0.1 N sodium hydroxide.

Subsequently, a scale up of this reaction was carried out, using a mass of 200 mg of ipolamide in each reaction, aiming to obtain a larger mass of hydrolysis product for subsequent isolation of the compounds of interest. Despite the increase in the mass of ipolamide, the time reaction and the volumes of hydrochloric acid and sodium hydroxide solutions were maintained.

### 4.2.1 L/L Partition of the Reaction Medium

After carrying out the hydrolysis reaction of the hypotonic amide, the reaction medium was subjected to extraction, performing a L/L partition with 15 mL of ethyl acetate. The organic fraction, which was isolated under N₂. Subsequently, the organic fraction was dissolved in water:acetonitrile (8:2), filtered through a PTFE membrane 0.22 μmean analyzed by LC-DAD, under the chromatographic conditions described in the evening 4.3.2.

### 4.3 Analysis of the Hydrolysis Product of Hydroxypropylamide by LC-DAD

### 4.3.1 Sample Preparation

After the hydrolysis reaction of the ipolamide, 250 μL of acetonitrile was added to the hydrolyzed reaction medium. Then, a 1 mL aliquot of the reaction medium was filtered through a 0.22 μm PTFE membrane and analyzed by LC-DAD.

### 4.3.2 LC-DAD Chromatographic Conditions

Column: Zorbax Eclipse XDB-C18 (4.6×15 0 mm, 5 μm);
Mobile phase A: 0.1% formic acid (A): acetonitrile (B) (see Table 5 below);
Flow: 1.2 mL/min;
Oven temperature: 25° C.;
Injector temperature: 20° C.;
Injection volume: 10 μL.

TABLE 5
Elution gradient used in LC-DAD analysis.

| t(min) | % B |
|---|---|
| 0 | 5 |
| 8 | 17 |
| 10 | 17 |
| 15 | 24.5 |
| 22 | 24.5 |
| 39 | 50 |
| 44 | 95 |
| 47 | 95 |
| 48 | 5 |
| 60 | 56 |

### 4.4 Analysis of the Hydrolysis Product of Hydroxypropylamide by LC-MS

### 4.4.1 Sample Preparation

An aliquot of the hydrolysis reaction product (reaction medium) was diluted 50 times with 0.1% formic acid: acetonitrile HPLC (9.5:0.5) diluent. This sample was filtered in a 0.22 μm PTFE membrane.

### 4.4.2 LC-MS Chromatographic Conditions

The chromatographic conditions used in LC-MS analysis are presented in item 4.3.2.

### 4.4.3 ESI-MS Parameters

Declustering Potential (DP): 40;
Entrance Potential (EP): 10;
Collision Energy (CE): 5;
Curtain Gas (CUR): 25;
Collision Gas (CAD): Medium;
Ion Spray Voltage (IS): 5000;
Temperature (TEM): 550° C.;
Ion Source Gas 1 (GS1): 45;
Ion Source Gas 2 (GS2): 45

### 4.5 Fractionation of the Ipolamide Hydrolysis Product by LC-UV on a Semipreparative Scale

### 4.5.1 Sample Preparation

The ipolamide hydrolysis product was stored in a freezer at −20° C. and subsequently subjected to drying by freeze-drying.

The dried sample was fractionated by LC-UV on a semipreparative scale, and injections were performed at concentrations between 15 and 25 mg/ml of this sample, using water:acetonitrile grade HPLC (8:2) as diluent. Before each injection, the sample was filtered through a 0.45 μm PTFE membrane filter.

A mass of 406 mg of sampled product was used in this ipolamide hydrolysis product fractionation process.

### 4.5.2 LC-UV Semipreparative Scale Chromatographic Conditions

Column: Shim-Pack C18, Shimadzu, dimensions 250 mm×20 mm, 5 μm;
Mobile phase: 0.1% formic acid (A): acetonitrile (B) gradient (Table 6);
Flow: 9.4 mL/min;
Injection volume: 2 mL.

TABLE 6
Elution gradient used in LC-UV semipreparative fractionation.

| t(min) | % B |
|---|---|
| 0 | 10 |
| 10 | 18 |
| 30 | 22.5 |
| 32 | 27 |
| 62 | 32 |
| 64 | 100 |
| 68 | 100 |
| 70 | 10 |
| 80 | 10 |

### 4.5.3 Analysis of the Fractions Obtained in LC-UV Fractionation on a Semipreparative Scale by LCDAD

### 4.5.3.1 Sample Preparation

The collected fractions were concentrated in a desiccator to eliminate the organic solvent. Then, the fractions were stored in a freezer at −20° C. and subsequently subjected to drying by freeze-drying.

Subsequently, samples of each fraction were solubilized in acetonitrile, filtered through a 0.22 μm PTFE membrane filter and analyzed by LC-DAD.

### 4.5.3.2 Chromatographic Conditions

The chromatographic conditions for LC-DAD analysis of the fractions are described in item 4.3.2.

### 4.5.4 NMR Analysis

The fractions collected in the semipreparative scale LC-UV fractionation were analyzed by NMR (NMR of ¹H-400 and 500 MHz and ¹³C NMR-100 and 125 MHz), using methanol solvents deuterated (CD₃OD) and deuterated chloroform (CDCl₃).

### 5 RESULTS AND DISCUSSION

### 5.1 Analysis of the Ipolamide Hydrolysis Product by LC-DAD

The hydrolysis product was analyzed by LC-DAD, using a method identical to that described in the document sent by Applicant.

The chromatographic profile obtained was compared with the chromatogram obtained with the chromatogram presented in the submitted document. Despite the non verification of total correspondence between the chromatographic profiles, peaks possibly corresponding to compounds 1 (Tr=8.41) and 5 (Tr=17.19 min) (FIG. 6) were suggested.

### 5.2 Analysis of the Ipolamide Hydrolysis Product by LC-MS

In the analysis of the hydrolysis product by LC-MS, it was noted that the ions of greater intensity MS spectrum of the peak with Tr=8.41 min are the same as those observed in the MS spectrum of the compound 1, provided by Applicant (see FIG. 7).

Furthermore, the ion m/z 249 [M+Na]⁺ was visualized in the peak spectrum with Tr=8.41 min, which corresponds to the sodiated molecule in the suggested structures for compound 1, in the report provided by Applicant (FIG. 7).

It was verified that the ions with the highest intensity in the MS spectrum of the peak with Tr=8.92 are the same observed in the MS spectrum of the compound 2, reported by Applicant (FIG. 7).

Furthermore, the ion m/z 249 [M+Na]⁺ was visualized in the peak spectrum with Tr=8.92, which corresponds to a sodiated molecule of a possible isomer of the compound 1 (FIG. 8).

The MS spectrum of the peak with Tr=17.19 min showed similarity with the spectrum of the compound 5 present in the report sent by Applicant (FIG. 9).

The m/z 203 [M+Na]⁺ ion was observed in the MS peak spectrum with Tr=17.19 min, which corresponds to the sodiated molecules suggested for the compound 5, in the report presented by Applicant (FIG. 9).

In view of the data obtained in the analyses by LC-DA and LC-MS, the rationing of the product ipolamide hydrolysis was directed towards isolation of peaks with Tr=8.41, Tr=8.92 and Tr=17.19 minutes.

### Analysis of the Organic Fraction of the Reaction Medium by LC-DAD

In the chromatographic profile of the ethyl acetate fraction, obtained by L/L partition of the product hydrolysis, a low intensity of the peaks corresponding to the ipolamide hydrolysis products in relation to the chromatographic profile of the reaction medium (FIG. 10) was verified. This result shows that there was no efficient extraction of the substances of interest by the organic solvent.

Given this observation, it was decided to carry out the fractionation from the lyophilized reaction medium, without carrying out the extraction step with organic solvent.

### 5.4 Fractionation of the Ipolamide Hydrolysis Product of by LC-UV on Semi-Preparative Scale

In the fractionation of the ipolamide hydrolysis product by LC-UV on a semi-preparative scale, seven fractions were collected and dried by lyophilization (FIG. 11).

Table 7 shows the masses of the dried fractions obtained in the LC-UV fractionation on a semi-preparative scale and mass yields of these fractions, considering the product mass of ipolamide hydrolysis subjected to fractionation.

The fractions were analyzed by LC-DAD under the same chromatographic conditions used for obtaining the chromatographic profile of the hydrolysis product. The chromatographic purities of the peaks majorities of each fraction were determined at 260 nm. The chromatograms relating to the analyses for determining the purity of the fractions obtained in the fractionation by semipreparative LC-UV are presented in ANNEX 1 below.

TABLE 7
Fractions collected in the fractionation of the ipolamide
hydrolysis product by LC-UV on semi-preparative scale.

| | | | | Purity % |
|---|---|---|---|---|
| Fraction | Tr (min) | Mass (mg) | Yield % ^{a} | (LC-DAD, 260 nm) |
| 1 | 8.82 | 3.6 | 0.88 | 97.72 |
| 2 | 8.91e9.91 | 3.4 | 0.83 | b |
| 3 | 9.02 | 3.4 | 0.83 | 91.96 |
| 4 | 13.34 | 2.1 | 0.51 | 95.33^{c}e46.78^{d} |
| 5 | 15.50 | 3.6 | 0.88 | 81.95 |
| 6 | 17.66 | 3.0 | 0.74 | 90.25 |
| 7 | 19.78 | 3.6 | 0.88 | 89.52 |

^{a} Calculated on the mass of the hydrolysis product subjected to fractionation (406 mg).
b More than one major peak was observed in the sample and their chromatographic purities were not determined.
^{c}Purity in the first analysis by LC-DAD.
^{d}Purity after 10 days of storage in the refrigerator.

### 5.4.1 Identification of the Fractions Obtained in LC-UV Fractionation on Semi-Preparative Scale by LC-DAD

The chromatographic profiles of the fractions, obtained by LC-DAD, were compared to those chromatograms of the reaction medium, with the aim of evaluating the presence of peaks of interest in these fractions. Subsequently, the fractions were analyzed by NMR. The NMR spectra ¹H, ¹³C, DEPT 135° and 2D NMR (HMQC and HMBC) of these fractions are presented in ANNEX 2 of this report.

### 5.4.2 Fraction 3

The chromatographic profile of fraction 3 (LC-DAD, 260 nm) showed a major peak with retention corresponding to the compound 2, reported in the report sent by Applicant (FIG. 12).

¹H, ¹³C, DEPT 135° NMR and NMR 2D (HMQC and HMBC) data were obtained from fraction 3. Interpretation of signals observed in ¹H and ¹³C NMR spectra, as well as the observed correlations in the 2D NMR spectra suggest that the majority peak of fraction 3 corresponds to the structure presented in FIG. 13. It was observed in the hydrogen spectrum the singlet with δ 3.70, corresponding to the methyl hydrogens of C12 methoxyl (Table 8). It was also observed a singlet at δ 1.27, which is related to the methyl hydrogens of position 10. Signs were verified with δ 7.52 and 6.04, which refer to the allylic hydrogens of positions 2 and 5, respectively. Thus, the molecule has two positions with sp² carbons. The signs with δ 2.61 and 2.37 correspond to the methylene hydrogens of position 6, in which the ¹³C NMR data confirm that C6 corresponds to the sole CH₂ of the molecule. It is noticed that the methinic hydrogens of the positions 8 and 9 exhibit chemical shifts of δ 2.61 and 5.06, respectively. In the spectrum of ¹³C NMR, the signal with δ 168 is visualized, which corresponds to the carbonyl of the molecule (C11). NMR data also allow the assignment of signals from the quaternary carbons at positions 3, 4 and 7 of the structure (Table 8).

The suggested structure for the majority peak of fraction 3 is compatible with the molecules referring to the ion m/z 249 [M+Na]⁺, which was observed both in the mass spectrum of compound 2, obtained in the LC-MS analyses in this study, as in the spectrum of compound 2 sent by Applicant. Thus, the isolated substance has a molecular mass of 226 Da, which is compatible with the mass of compound 1 and compound 2 isomers reported by Applicant.

The structural data obtained suggest that the majority peak of fraction 3 is a hydrolysis product of the glycosidic bond at position 9 of the ipolamide, which results in the loss of a glucose residue. In addition, there is also a dehydration reaction from the ipolamide and bond formation π between C4 and C5.

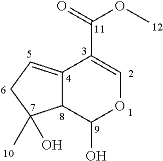 [SMILES: COC(=O)C1=COC(O)C2C1=CCC2(C)O]

TABLE 8
NMR data of the major peak of fraction 3.

| | | ¹³C | | |
|---|---|---|---|---|
| | ¹H | NMR | DEPT | |
| Position | NMR[δ; m; J (Hz)] | (δ) | 135° | HMBC |
| 1 | — | — | — | |
| 2 | 7.52; s | 156.9 | CH | C3; C4; C9; C11 |
| 3 | — | 130.3 | C | |
| 4 | — | 107.4 | C | |
| 5 | 6.04; m | 122.9 | CH | |
| 6 | 2.61; m | 49.6 | CH₂ | C3; C5; C7; C8; C10 |
| | 2.37; dd; 2.4e16.2 | | | |
| 7 | — | 81.1 | C | |
| 8 | 2.61; d; 9.8 | 57.2 | CH | C3; C5; C7; C10 |
| 9 | 5.06; d; 9.8 | 99.9 | CH | |
| 10 | 1.27; s | 23.2 | CH₃ | C6; C7; C8; C9 |
| 11 | — | 168.0 | C | |
| 12 | 3.70; s | 51.6 | CH₃ | C11 |

### 5.4.3 Fraction 1

The majority peak of the fraction 1 (LC-DAD, 260 nm) showed retention and intensity consistent with compound 1, reported in the report sent by Applicant (FIG. 14).

¹H, ¹³C, DEPT 135° NMR and 2D NMR (HMQC and HMBC) data were obtained from fraction 1, wherein the interpretation of the data from these experiments was associated with the NMR data from the fraction 3 and they suggest that the majority peak of the fraction 1 corresponds to the structure shown in FIG. 15.

Great similarity was observed between NMR spectra ¹H and ¹³C of the fractions 1 and 3. Based on this result and in the 2D NMR data, the signals of the hydrogen and carbon of the positions 2 to 12 in the structure were assigned. Additionally, the signals δ 4.78, 3.83, 3.72 and 3.34 were visualized in the ¹H NMR spectrum, which correspond to the hydrogens of a glucose molecule linked to position 9 of the structure (Table 9). Based on this information, it is suggested that the structure corresponding to the majority peak of fraction 1 is a substance formed from the dehydration reaction of ipolamide and, consequently, formation of TT bonds between C4 and C5 (FIG. 15).

It is noteworthy that signals observed in the NMR spectra of compound 1, presented by Applicant, were not observed in the NMR spectra of fraction 1.

TABLE 9
NMR data of the major peak of fraction 1.

| | | ¹³C NMR | DEPT | |
|---|---|---|---|---|
| Position | ¹H NMR[δ; m; J (Hz)] | (δ) | 135° | HMBC |
| 1 | — | — | — | |
| 2 | 7.51; s | 155.9 | CH | C3, C4, C9, C11 |
| 3 | — | 107.9 | C | |
| 4 | — | 129.5 | CH₂ | |
| 5 | 6.09; m | 123.7 | CH | C6, C7, C8 |
| 6 | 2.60 | 49.7 | CH₂ | C4, C5, C7, C8 |
| | 2.37; m | | | |
| 7 | — | 81.3 | CH₃ | |
| 8 | 2.76; dl; 9.8 | 55.5 | CH | C4, C6, C9 |
| 9 | 5.37; d; 9.8 | 100.5 | CH | C4, C7, C8, C1′ |
| 10 | 1.35; s | 23.1 | CH₃ | C5, C7, C8, C9 |
| 11 | — | 167.7 | C | |
| 12 | 3.70; s | 51.7 | CH₃ | C3, C11 |
| 1′ | 4.78; d; 7.8 | 100.5 | CH | C9 |
| 2′ | 3.34; m | 71.3 | CH | |
| 3′ | 3.34; m | 74.6 | CH | |
| 4′ | 3.34; m | 78.4 | CH | |
| 5′ | 3.34; m | 77.7 | CH | |
| 6′ | 3.83; m | 62.5 | CH₂ | |
| | 3.65; m | | | |

### 5.4.4 Fraction 4

Fraction 4 was analyzed by LC-DAD after fractionation and subjected to NMR analysis. After these analyses, fraction 4 was stored in the refrigerator for 10 days and, after this period, analyzed again by LC-DAD.

In the first analysis by LC-DAD, the sample showed a major peak with Tr=13.4 min. In subsequent analysis, the appearance of a peak with retention time equivalent to the major component of fraction 1 (Tr=8.72 min), which suggested degradation of the major peak of fraction 4 (FIG. 16A).

1D NMR spectra were obtained (¹H) and 2D (HMQC and HMBC) of fraction 4. NMR data of this fraction shows the presence of signals related to methyl, methoxy, sp² carbons, as well as signals related to glucose. This information was compared to the NMR data of fractions 1 and 3 and, thus, the suggested structure for fraction 4 corresponds to a derivative of ipolamide with a double bond between carbons C3 and C4, formed from the dehydration of ipolamide, as well as the presence of an OH group in C2. Furthermore, the NMR data of this fraction show the presence of glucose in the molecule (FIG. 17).

However, 1D and 2D NMR spectra showed the occurrence of signals from another molecule in the sample, the compilation of the data from fraction 4 with the NMR data from fraction 1 suggesting that the majority constituent of fraction 1 is also present in fraction 4. This observation is corroborated by the appearance of a peak with Tr=8.72 min in the second analysis of fraction 4 by LC DAD (FIG. 16B).

Thus, two substances formed from the ipolamine degradation were verified in fraction 4, being named substance 4 and substance 1, which have Tr of 13.4 and 8.72 min, respectively (Tables 10 and 11, FIGS. 17 and 18).

It is worth noting that substance 1 presents a conjugated diene (C2=C3 and C4=5), which is susceptible to the 1,4 addition reaction, forming substance 4.

TABLE 10
NMR data of substance 4 (Tr = 13.4 min).

| Position | ¹H NMR[δ; m; J (Hz)] | ¹³C NMR (δ) | HMBC |
|---|---|---|---|
| 1 | — | — | |
| 2 | 7.52; s | 155.8 | C3, C4, C9 |
| 3 | — | 106.4 | |
| 4 | — | 128.7 | |
| 5 | 6.08; m | 122.8 | |
| 6 | 2.35; m | 48.2 | |
| | 2.66; m | | |
| 7 | — | 79.0 | |
| 8 | 2.67; dl; 98 | 54.8 | |
| 9 | 5.40; d; 9.8 | 98.3 | C1′ |
| 10 | 1.28; s | 21.9 | C6, C7, C8 |
| 11 | — | 166.29 | |
| 12 | 3.64; s | 50.3 | C11 |
| 1′ | 4.82; d; 9.8 | 98.4 | C9 |
| 2′ | 3.22-3.49; m | 69.0-76.0 | |
| 3′ | 3.22-3.49; m | 69.0-76.0 | |
| 4′ | 3.22-3.49; m | 69.0-76.0 | |
| 5′ | 3.22-3.49; m | 69.0-76.0 | |
| 6′ | 3.22-3.49; m | 69.0-76.0 | |

TABLE 11
NMR data of substance 1 (Tr = 8.72 min).

| Position | ¹H NMR[δ; m; J (Hz)] | ¹³C NMR (δ) | HMBC |
|---|---|---|---|
| 1 | — | — | |
| 2 | 5.52; sl | 96.1 | C3, C4 |
| 3 | — | 121.5 | |
| 4 | — | 158.5 | |
| 5 | 2.75; m | 28.2 | |
| 6 | 1.76; m | 38.3 | C4, C7, C8 |
| | 1.83; m | | |
| 7 | — | 77.4 | |
| 8 | 2.67; d; 8.6 | 54.9 | C7, C9 |
| 9 | 5.26; d; 8.6 | 91.7 | C2, C1′ |
| 10 | 1.16; s | 20.3 | C6, C7, C8 |
| 11 | — | 165.1 | |
| 12 | 3.71; s | 50.3 | |
| 1′ | 4.69; d; 8.6 | 98.3 | C9 |
| 2′ | 3.22-3.49; m | 69.0-76.0 | |
| 3′ | 3.22-3.49; m | 69.0-76.0 | |
| 4′ | 3.22-3.49; m | 69.0-76.0 | |
| 5′ | 3.22-3.49; m | 69.0-76.0 | |
| 6′ | 3.22-3.49; m | 69.0-76.0 | |

### 5.4.5 Fraction 5

Comparison of the chromatograms of fraction 5 (LC-DAD, 260 nm) and the reaction product is shown in FIG. 17.

1D (¹H) and 2D (HMQC and HMBC) NMR spectra were obtained for fraction 5. The signals observed in the spectra were compared to NMR data previously obtained for fractions 1, 3 and 4. In the NMR spectrum signs of methyl hydrogens with δ 2.00 and 2.15 ppm were observed, as well as signs of methyl hydrogens of methoxyl group with δ 3.70 and 3.78 ppm.

Furthermore, the 2D NMR spectra suggested the presence of sp² carbons, as well as other signals that were compared to the correlations observed in fractions 1, 3 and 4 and made it possible to suggest that there is the presence of a ipolamide derivative in fraction 5. A signal with δ 208.1 ppm was also seen on the HMBC contour map of fraction 5, which is compatible with an acetone carbonyl. However, the sample showed a large number of signs, which indicates that it is a mixture of substances. Given these results, it was not possible to elucidate the components of this mixture.

### 5.4.6 Fraction 6

Comparison of the chromatographic profile (LC-DAD260 nm) of fraction 6 with the chromatogram of the ipolamide hydrolysis product is shown in FIG. 20.

The ¹H NMR spectrum of fraction 6 was obtained, observing the presence of methoxyl methyl hydrogens and methyl and allylic hydrogens in the spectrum, as observed in the other ¹H NMR spectra obtained in this study. As described for fraction 5, the presence of interfering signs did not allow structural determination of the major component of this fraction.

### 5.4.7 Fraction 7

The majority peak of fraction 7 showed retention similar to the peak possibly related to compound 5, presented in the Applicant report (FIG. 21).

1D (¹H) and 2D (HMQC and HMBC) NMR spectra of fraction 7 were obtained. In the analyses of these In the spectra, signals of methyls, methoxyls, as well as signals corresponding to glucose were identified, as observed in other ipolamide derivatives in the fractions analyzed in this study.

However, the NMR data suggest a mixture of substances, which did not allow the structural elucidation of the mixture constituents.

### 6 CONCLUSIONS

The ipolamide hydrolysis product was analyzed by LC-DAD and LC-MS, using the chromatographic conditions characteristics described in the report sent by Applicant. From the comparison between the chromatography profile obtained in this study with that presented in the previous study provided by Applicant, combined with the m/z information relating to each chromatographic peak, the peaks that were suggested would possibly correspond to compounds 1 and 5 described in the report sent by Applicant and targets of this study.

Subsequently, different chromatographic techniques were performed to fractionate the product of the hydrolysis of ipolamide, and fractionation by LC-UV on semipreparative scales allowed the isolation of major products obtained from hypotonic amide. The fractions obtained were analyzed by LC-DAD, and their respective chromatographic purities were determined.

These fractions were also analyzed by 1D (¹H and ¹³C) and 2D (HMQC and HMBC) NMR. None of the spectra obtained showed signals that could be attributed to the molecules identified in the report provided by Applicant. The compilation of the spectral data obtained made it possible to determine the structure of three isolated substances, indicated below, which are, in general, the result of dehydration reactions and glucose loss.

### ANNEX 1

Chromatograms (LC-DAD, 260 nm) of the fractions obtained by fractionating the ipolamide hydrolysis product by LC-UV on semi-preparative scale.

### ANNEX 2

NMR spectra of the fractions collected in the fractionation of the ipolamide hydrolysis product by LC-UV semi-preparative scale as set forth in the attached FIGS. 22-49.

Those skilled in the art will appreciate the knowledge presented herein and may reproduce the invention in the embodiments presented and in other embodiments, falling within the scope of the appended claims.

What is claimed is:

1. A compound derived from ipolamiide of general formula (I), (II) and/or (III) as follows: 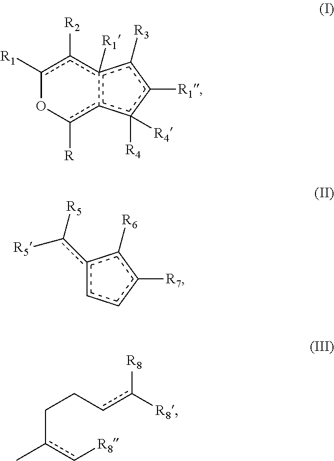
wherein R corresponds to H, OH, OGlyc (Glycoside); R₁, R_{1′}, R_{1″} correspond to H, OH; R₂ corresponds to H, COOH, COOCH₃, CH₃, CHO; R₃ corresponds to H, OH, CH₃; R₄, R_{4′} correspond to H, OH, CH₂OH, CH₃; R₅, R_{5′} correspond to H, CH₃, COOCH₃, CHO, CH₂OH; R₆ corresponds to CHO, COOH, COOCH₃; R₇ corresponds to H, CH₃; R₈, R_{8′}, R_{8″} correspond to CHO, CH₃, CH₂OH, COOH and the dashed bonds represent single (C—C) or double (C═C) bonds between carbons (up to two double bonds per structure), and wherein R₁ may be absent or correspond to H or OH.

2. The compound according to claim 1 wherein said compound has immunosuppressive activity.

3. The compound according to claim 1 wherein the compound comprises at least one compound having the general formula (I), (II) and/or (III) as follows:

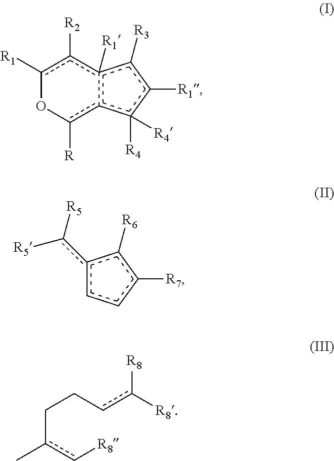

4. A pharmaceutical composition comprising the compound according to claim 1 and a pharmaceutically acceptable vehicle.

5. The pharmaceutical composition according to claim 4 wherein the compound comprises at least one compound having the general formula (I), (II) and/or (III) as follows:

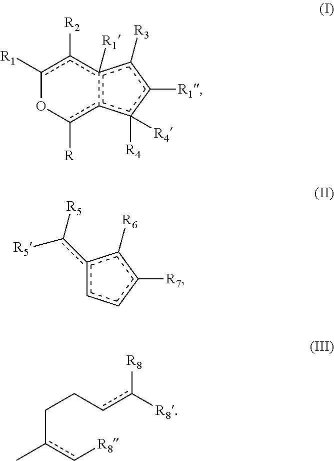

6. The pharmaceutical composition according to claim 4, further comprising the ipolamiide compound.

7. The pharmaceutical composition according to claim 4 wherein the compound comprises at least one of the following compounds:

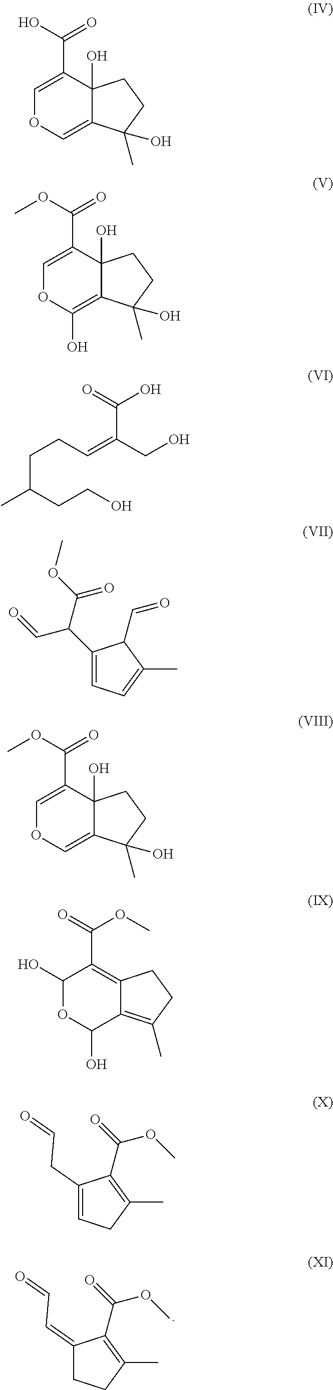

8. The pharmaceutical composition according to claim 4, further comprising at least one of the following compounds:

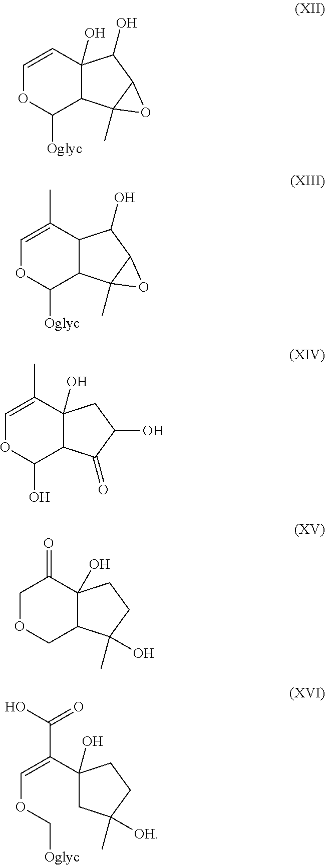

9. A method of treating an immunological disorder comprising administering to a patient in need thereof an effective amount of the compound according to claim 1.

10. The method of treatment according to claim 9, wherein the treatment is for patients with vitiligo.

11. A method of treating an immunological disorder comprising administering to a patient in need thereof an effective amount of the pharmaceutical composition according to claim 4.

12. The method of treatment according to claim 11, wherein the treatment is for patients with vitiligo.

13. A method of treating an immune disorder comprising administering to a patient in need thereof an effective amount of at least one compound of general formula (I), (II) and/or (III) below in an amount sufficient to provide immunosuppressive effect, wherein general formula (I), (II) and/or (III) are as follows: 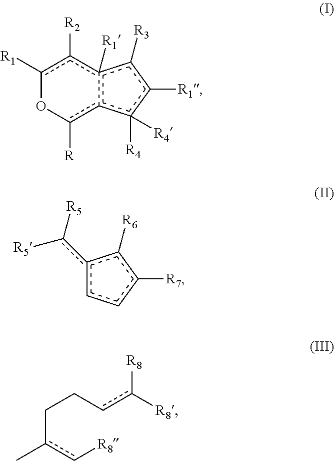
wherein R corresponds to H, OH, OGlyc (Glycoside); R₁, R_{1′}, R_{1″} correspond to H, OH; R₂ corresponds to H, COOH, COOCH₃, CH₃, CHO; R₃ corresponds to H, OH, CH₃; R₄, R_{4′} correspond to H, OH, CH₂OH, CH₃; R₅, R_{5′} correspond to H, CH₃, COOCH₃, CHO, CH₂OH; Re corresponds to CHO, COOH, COOCH₃; R₇ corresponds to H, CH₃; R₈, R_{8′}, R_{8″} correspond to CHO, CH₃, CH₂OH, COOH and the dashed bonds represent single (C—C) or double (C═C) bonds between carbons (up to two double bonds per structure), and wherein R₁ may be absent or correspond to H or OH.

14. A process for producing active compounds comprising subjecting at least one ipolamiide compound to heating at high temperatures in a suitable solvent for a time sufficient to obtain the ipolamiide derivatives of formula (I), (II) and/or (III) as follows: 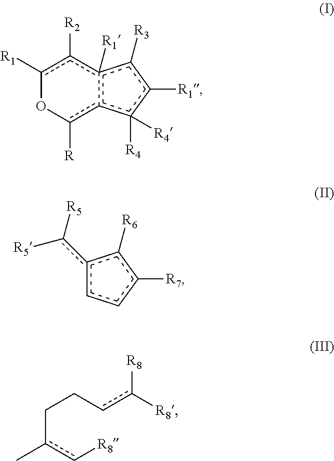
wherein R corresponds to H, OH, OGlyc (Glycoside); R₁, R_{1′}, R_{1″} correspond to H, OH; R₂ corresponds to H, COOH, COOCH₃, CH₃, CHO; R₃ corresponds to H, OH, CH₃; R₄, R_{4′} correspond to H, OH, CH₂OH, CH₃; R₅, R_{5′} correspond to H, CH₃, COOCH₃, CHO, CH₂OH; R₆ corresponds to CHO, COOH, COOCH₃; R₇ corresponds to H, CH₃; R₈, R_{8′}, R_{8″} correspond to CHO, CH₃, CH₂OH, COOH and the dashed bonds represent single (C—C) or double (C═C) bonds between carbons (up to two double bonds per structure), and wherein R₁ may be absent or correspond to H or OH.

15. The process according to claim 14, wherein the solvent is water.

16. The process according to claim 14, further comprising obtaining concentrations of the compounds of formula (I), (II) and/or (III) of from about 0.5% to about 45% in a mixture.

17. The process according to claim 14, wherein the high temperatures comprise temperatures of 35° C. and above.

18. The process according to claim 17, wherein the high temperatures comprise temperatures between 35° C. and 165° C.

19. The process according to claim 14, further comprising hydrolysis, acid hydrolysis or basic hydrolysis.

20. An extract obtained from the plants of the genus Stachytarpheta comprising at least one compound of general formula (I), (II) and/or (III) as follows: 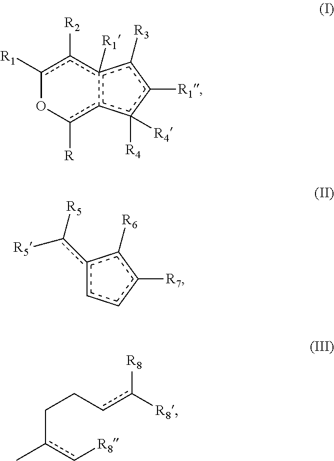
wherein R corresponds to H, OH, OGlyc (Glycoside); R₁, R_{1′}, R_{1″} correspond to H, OH; R₂ corresponds to H, COOH, COOCH₃, CH₃, CHO; R₃ corresponds to H, OH, CH₃; R₄, R_{4′} correspond to H, OH, CH₂OH, CH₃; R₅, R_{5′} correspond to H, CH₃, COOCH₃, CHO, CH₂OH; R₆ corresponds to CHO, COOH, COOCH₃; R₇ corresponds to H, CH₃; R₈, R_{8′}, R_{8″} correspond to CHO, CH₃, CH₂OH, COOH and the dashed bonds represent single (C—C) or double (C═C) bonds between carbons (up to two double bonds per structure), and wherein R₁ may be absent or correspond to H or OH.

21. A process for producing an extract enriched with the compound of claim 1 comprising:
a) selecting input vegetal biomass with a content of ipolamiide between 2.5% and 3.5% obtained from plants of the genus Stachytarpheta;
b) submitting the selected vegetal biomass from a) to oven drying at a temperature between 40 to 80° C., until obtaining a humidity stabilization between 10 to 12%;
c) milling the vegetal biomass; and
d) performing the extraction of the vegetal biomass by:
i. heating the vegetal biomass at a temperature between 70 to 100° C., with constant stirring;
ii. macerating the vegetal biomass at room temperature; and
iii. heating the vegetal biomass with temperature between 70 to 100° C.

22. The process according to claim 21, further comprising:
iv. filtering and concentration of the extract; and
v. drying in Spray Dryer, during 1 to 60 seconds, with an inlet temperature between 155 and 165° C. and an outlet temperature between 85 to 95° C., coupled to a dehumidifier.

23. The process according to claim 21, further comprising predicting the theoretical content of ipolamiide and derivatives in the extract from the actual content of ipolamiide in the input vegetal biomass using Equation I as shown below:
$$\% Theoretical content of ipolamiide and derivatives in the extract = \% Actual content of ipolamiide in the vegetal biomass \times DER / ( < actual content of ipolamiide and derivatives in the extract / actual content of ipolamiide in the input vegetal biomass > ) \pm standard deviation .$$

24. An extract enriched with compounds obtained by the process according to claim 21.
